(19) United States
(12) Patent Application Publication
(10) Pub. No.: US 2008/0280848 A1
(43) Pub. Date: Nov. 13, 2008

(54) Structures of Active Guide Rna Molecules and Method of Selection

(76) Inventors: Volker Patzel, Hanau (DE); Stefan H. E. Kaufmann, Berlin (DE)

Correspondence Address: NIXON PEABODY, LLP, 401 9TH STREET, NW, SUITE 900, WASHINGTON, DC, 20004-2128, US

(21) Appl. No.: 12/091,937

(22) PCT Filed: Oct. 27, 2006
(86) PCT No.: PCT/EP2006/010372

§ 371 Date: Apr. 28, 2008

(30) Foreign Application Priority Data
Oct. 28, 2005 (EP) 05023704.9
Oct. 28, 2005 (EP) 05023706.4

(51) Int. Cl. A61K 31/7088; C07H 21/02; C12Q 1/68; A61P 43/00; C12P 19/34

(52) U.S. Cl. 514/44; 536/23.1; 435/91.1; 435/6

(57) ABSTRACT

The present invention relates to methods and compositions for modulating RNA silencing efficiency by providing selective RISC (RNA-induced silencing complex) formation. The present invention also relates to methods for identifying nucleic acids and/or determining their structures.

The present invention relates to methods and compositions for modulating RNA silencing efficiency by providing guide RNA molecules with increased RNA silencing activity. The present invention further relates to methods for identifying nucleic acids and/or determining their structures.

In mammalian systems, RNAi is mainly triggered by siRNAs and microRNAs (miRNAs)¹⁻⁴. SiRNA and miRNA duplexes are composed of complementary RNA of preferably 21-23 nucleotides (nts) in length with sense and antisense orientation to the mRNA target. In siRNA duplexes, sense- and antisense-siRNA (as-siRNA) are perfectly base-paired. mRNA duplexes exhibit imperfect pairing between the mature miRNA (antisense) and the opposing strand termed miRNA (sense). One of the two strands, the guide strand, is included into the RISC, whereas the other strand, the passenger strand, is excluded and destroyed. Only if the antisense strand is chosen as guide, it directs activated RISC to the mRNA target inducing gene silencing. RISC is a multiprotein complex containing as core a protein of the Argonaute (Ago) family. MiRNA-associated RISC contains Ago-1, 2, 3 or 4, whereas siRNA-induced mRNA cleavage is exclusively associated with Ago-2-containing RISC⁵.

In mammalian cells, siRNA-triggered RNAi starts with formation of the RISC-loading complex (RLC) including siRNA duplex recognition and definition of guide and passenger strand. Subsequent steps encompass duplex unwinding, RISC formation and activation, mRNA targeting, cleavage, and release of the cleaved target sequence prior to targeting of further mRNA molecules^{6,7}. Lower thermodynamic duplex stabilities at the 5′ antisense compared to the 5′ sense terminus favor selection of as-siRNAs as guide strands and, thus, formation of silencing competent RISCs⁸⁻¹⁰. Specific base preferences and GC contents, the absence of internal repeats, and accessible target sites were reported to favor siRNA activities¹¹⁻¹⁸. However, the meaning of many of these correlations for the silencing pathway and, thus siRNA design remains unclear.

Ribonucleic acids (RNA) can adopt the coding potential (genotype) of deoxyribonucleic acids and, moreover, some coding or non-coding RNA molecules comprise phenotypes (functions) such as nuclease resistance, RNA processing, transport, and a.o. inhibition of gene expression. The later class of molecules comprises small interfering RNAs (siRNA), antisense RNAs (asRNA), ribozymes, and indirectly target sites of messenger RNA (mRNA) that are accessible to complementary nucleic acids or other drugs.

RNA function is directly related to RNA primary, secondary, and or tertiary structure. RNA primary structures (sequences) are easiest accessible but not suitable to explain most RNA functions, on the other hand RNA tertiary structures are best suitable to explain functions of RNA but are only hardly accessible.

RNA secondary structures represent an intermediate status and are accessible by experimental and computational procedures with more or less success. Although, computational methods bypass all experimental limitations, individual predictions of RNA secondary structures frequently lead to contradictionary results and cannot be correlated to RNA function.

Small interfering ribonucleic acids (siRNA) are the mediators of messenger RNA (mRNA) degradation in the process of RNA interference (RNAi). SiRNAs are short RNA duplexes composed of partly overlapping or blunt sense and antisense siRNAs. Target mRNA structures are described to play a role in RNAi, however, the described methods for identification of suitable target structures are rather inefficient.

Algorithms for RNA secondary structure prediction do not allow systematic analysis and characterization of RNA secondary structures, do not include functions to calculate structure parameters that are related to RNA function and, thus, are not suitable for identifying structure function relationships and selection or prediction of above defined functional RNA molecules. Additionally, the existing algorithms are not automated and not parallelizable, and, thus not suitable for RNA structure prediction and analysis in a high-throughput compatible manner.

A variety of important high-throughput technologies, such as DNA-chiptechnologie, functional genomics, and modern nucleic acid-based drug design strongly depend on functional RNA molecules. So far, only a fraction of siRNAs selected by state of the art procedures induces RNAi. As a consequence, for every single target sequence 2 to 4 siRNAs have to be designed and tested in order to achieve efficient down regulation of gene expression. Thus there is a strong need for reliable and flexible tools that allow the directed selection or design of functional RNA molecules, siRNA in particular.

This object is realized through the embodiments characterized in the attached patent claims 34 to 97. In particular, an algorithm was prepared which is suitable to reliably analyse and select functional RNA molecules or structures including inhibitory siRNAs.

The invention is based on data which demonstrate that selection of antisense or guide strand structures may lead to a modulated, e.g. an increased or reduced RNA silencing activity in target cells, organisms or cell-free systems.

A first aspect to the invention relates to a method for preparing a double stranded RNA molecule with target gene specific silencing activity, comprising the steps
(a) identifying a double stranded RNA molecule directed to the mRNA of a target gene, wherein said RNA molecule comprises:
   (i) a double stranded portion of 9-35 nucleotides and optionally at least one 3′-overhang,
   (ii) an antisense strand which has a sufficient degree of complementarity to the mRNA of the target gene for formation of an RNA-induced silencing complex (RISC), and accessible 5′- and 3′-ends which do not form stable intramolecular secondary structures,
   (iii) a sense strand which has a sufficient degree of complementarity to the antisense strand for interaction with a RISC, and
(b) preparing a double stranded RNA molecule or a precursor thereof or a DNA molecule encoding said RNA molecule or precursor.

A further aspect relates to a method for regulating the expression of a target gene in a cell, an organism or a cell-free system comprising the steps of:
(a) identifying a double stranded RNA molecule directed to the mRNA of a target gene, wherein said RNA molecule comprises:
   (i) a double stranded portion 9-35 nucleotides and optionally at least one 3′ overhang,
   (ii) an antisense strand which has a sufficient degree of complementarity to the mRNA of the target gene for formation of an RNA-induced silencing complex (RISC), and accessible 5′- and 3′-ends which do not form stable intramolecular secondary structures,
   (iii) a sense strand which has a sufficient degree of complementarity to the antisense strand for interaction with a RISC,
(b) preparing a double stranded RNA molecule or a precursor thereof or a DNA molecule encoding said RNA molecule or precursor and
(c) contacting said mRNA of the target gene with said double stranded RNA molecule under conditions under which target-specific nucleic acid silencing occurs.

Still a further aspect of the invention relates to a method for preparing a double stranded RNA molecule with target gene specific gene silencing activity, comprising the steps of:
(a) identifying a double stranded RNA molecule directed to the mRNA of a target gene, wherein said RNA molecule comprises:
   (i) a double stranded portion 9-35 nucleotides and optionally at least one 3′ overhang,
   (ii) an antisense strand which has a sufficient degree of complementarity to the mRNA of the target gene for formation of an RNA-induced silencing complex (RISC), and accessible 5′- and 3′-ends which do not form stable intramolecular secondary structures, and at least one wobble base pair (G-U, U-G) between the antisense strand and the target sequence,
   (iii) a sense strand which has a sufficient degree of complementarity to the antisense strand for interaction with a RISC, and
(b) preparing a double stranded RNA molecule or a precursor thereof or a DNA molecule encoding said RNA molecule or precursor.

The invention also relates to double stranded RNA molecules or precursors thereof or DNA molecules encoding said RNA molecules or precursors or compositions comprising these molecules.

Furthermore, the invention relates to a double stranded RNA molecule with target gene specific silencing activity comprising:
(a) a double stranded portion of 9-35 nucleotides and optionally at least one 3′-overhang
(b) an antisense RNA strand which has a sufficient degree of complementarity to the mRNA of the target gene for formation of an RNA-induced silencing complex (RISC), and accessible 5′- and 3′-ends which do not form stable intramolecular secondary structures, and
(c) a sense RNA strand which has a sufficient degree of complementarity to the antisense strand for interaction with a RISC, or a precursor thereof or a DNA molecule encoding the double stranded RNA molecule or the precursor thereof.

Furthermore, the invention relates to a double stranded RNA molecule with target gene specific silencing activity comprising a double stranded portion:
(a) an antisense RNA strand which has 9-35 nucleotides and optionally at least one 3′-overhang,
(b) a sufficient degree of complementarity to the mRNA of the target gene for formation of an RNA-induced silencing complex (RISC), accessible 5′- and 3′-ends which do not form stable intramolecular secondary structures, and at least one wobble base pair (G-U, U-G) between the antisense strand and the target sequence, and
(c) a sense RNA strand which has a sufficient degree of complementarity to the antisense strand for interaction with a RISC, or a precursor thereof or a DNA molecule encoding the double stranded RNA molecule or the precursor thereof.

The compounds and compositions of the present invention are suitable as reagents, diagnostics or medicaments.

The present invention relates to the field of RNA silencing which describes a gene regulatory mechanism that limits the transcript level by suppressing transcription, i.e. transcriptional gene silencing (TGS) or by activating a sequence-specific RNA degradation process (post-transcriptional gene silencing (PTGS)). PTGS includes translational attenuation and/or RNA interference. Three phenotypically different but mechanistically similar forms of RNAi, cosuppression or PTGS in plants, quelling in fungi, and RNAi in the animal kingdom, have been described. More recently, micro-RNA formation, heterochromatinization, etc., have been revealed as other facets of naturally occurring RNAi processes of eukaryotic cells. RNA silencing is mediated by RISC formation. An RISC may contain as a core different proteins of the Argonaute family.

According to the present invention, double stranded RNA molecules with RNA silencing activity are provided which interact with a RISC containing a determined species of Argonaute protein, e.g. Ago-1, Ago-2, Ago-3, Ago-4, PIWIL 1, PIWIL 2, PIWIL 3 or PIWIL 4, preferably Ago-1 or Ago-2. Preferably, the RISC is a mammalian RISC, e.g. a human RISC and the Argonaute proteins are mammalian, e.g. human proteins.

The double stranded RNA molecule with gene silencing activity comprises a double stranded portion of e.g. 9-35 nucleotides, preferably 14-25 nucleotides and more preferably 18-22 nucleotides and optionally at least one, e.g. one or two 3′ overhangs which have a length of e.g. 1-10, preferably 1-5, such as 1, 2, 3, 4 or 5 nucleotides.

The double stranded RNA molecule comprises an antisense strand which has a sufficient degree of complementarity to the mRNA of the target gene for RISC formation. For example, the degree of complementarity may be at least 50%, preferably at least 70% and more preferably at least 90%, e.g. 100% to the mRNA of a target gene. In this context, it should be noted that complementarity according to the present application is defined as comprising Watson-Crick base pairs, i.e. A-U, U-A, G-C and C-G base pairs and Wobble base pairs, i.e. G-U and U-G base pairs.

The double stranded RNA molecule also comprises a sense strand which has a sufficient degree of complementarity to the antisense strand to provide a double stranded RNA molecule which is suitable for interaction with a RISC. The sense strand and the antisense strand have usually a length between 9 and 40 nucleotides, preferably between 15 and 30 nucleotides and more preferably between 19 and 25 nucleotides.

According to the present invention, an increased silencing activity is achieved by selecting an antisense strand of a double stranded RNA molecule with accessible 5′- and 3′-ends which do not form stable intramolecular secondary structures, for example, the 5′- and 3′-ends of the antisense strand may have an unpaired conformation, an internal loop (il) conformation, e.g. a short, e.g. 1, 2 or 3 nt long paired conformation followed by one or several mismatches, a two-stem loop (2-sl) conformation, e.g. a conformation of two short, e.g. 1, 2 or 3 nucleotides long stem structures followed by loops, or other pseudo-paired structures.

The accessibility of the structure of the antisense strand may be determined by calculating the minimal Gibbs free energy. Preferably, the antisense strand has a minimal Gibbs free energy of about ≧0 kcal/mol, preferably of about ≧0.5 kcal/mol, more preferably of about ≧1.3 kcal/mol and most preferably of about ≧2.8 kcal/mol. The minimal free Gibbs energy may be calculated according to known methods, e.g. by an algorithm as described in Zuker and Stiegler (Nucleic Acids Res. 9 (1981), 133-148), which is incorporated herein by reference. A particularly preferred program is mfold 2.0. Alternatively, the accessibility of the 5′- and 3′-ends of the antisense strand may be determined by a partition function approach which gives base-pairing probabilities for a Boltzmann ensemble of secondary structures, e.g. a complete Boltzmann ensemble or a statistically unbiased sample of it. Preferred examples of a partition function approach are described in McCaskill (Biopolymers 29 (1990), 1105-1119) and Ding and Lawrence (Nucleic Acids Res. 31 (2003), 7280-7301), which are herein incorporated by reference. In an especially preferred embodiment, the antisense strand is substantially free from secondary structures and comprises a random coil structure.

The length of a 5′ accessible end is preferably at least two nucleotides. The length of a 3′ accessible end of an antisense strand is preferably at least 4 nucleotides, more preferably at least 5, 6, 7, 8, 9 or 10 nucleotides.

The antisense strand may comprise at least one Wobble base pair between the antisense strand and the target sequence. The Wobble base pair may be located in the antisense strand preferably at a position selected from positions 3, 4, 5, 6, 7, 8, 9, 10, 11, 12, 13, 14, 15, 16, 17, 18, 19, 20 or higher (when the 5′ position of the antisense strand is designated as position 1). By introducing Wobble base pairs, the accessibility of the 5′- and/or 3′-ends may be enhanced leading to an increased gene silencing activity.

In order to achieve selective interaction of a double-stranded RNA molecule with an RISC determined species of Argonaute protein, a predetermined degree of complementarity between sense and antisense strand may be selected. For example, in a double stranded RNA molecule which is intended to selectively interact with an Ago-2 containing RISC the sense strand is selected to have a degree of complementarity with the antisense strand of 100%, wherein complementarity comprises Watson-Crick base pairs and Wobble base pairs, e.g. the sense strand and the antisense strand have a 100% complementarity of Watson-Crick base pairs only or a 100% complementarity of Watson-Crick base pairs plus at least one Wobble base pair.

If, on the other hand, a double stranded RNA molecule which selectively interacts with a non Ago-2-containing RISC, e.g. an Ago-1 containing RISC is prepared, the sense strand is selected to have a degree of complementarity of less than 100% to the antisense strand, wherein complementarity comprises Watson-Crick base pairs and Wobble base pairs, e.g. only Watson-Crick base pairs or Watson-Crick base pairs and at least one Wobble base pair. Thus, the double stranded portion of the sense and antisense strand comprises at least one mismatch. If, for example, the antisense strand has a length of 1-23, preferably 20-22 nucleotides, the at least one mismatch is preferably located between position 13 and 17, more preferably between position 14 and 16 of the antisense strand (when the 5′ end of the antisense strand is designated as position 1).

The double stranded RNA may be an siRNA or an miRNA or a precursor thereof. The term “precursor” relates to an RNA species which is processed in the cell to a double stranded RNA with target-gene specific silencing activity. Preferred examples of precursors of siRNA molecules are small hairpin (sh) molecules, i.e. single stranded RNA molecules having a stem-loop structure wherein the stem corresponds to the double stranded RNA and the loop portion is cleaved off. Further examples of siRNA precursors are long double stranded RNA molecules which are processed within a cell, particularly an eukaryotic cell in order to give double stranded RNA molecules as indicated above.

Preferred examples of precursors of miRNA molecules are primary miRNA molecules or precursor miRNA molecules which are processed by Drosha or Dicer respectively to mature miRNA molecule comprising an antisense and a sense strand.

Further, the invention relates to DNA molecules encoding the double stranded RNA molecule or a precursor thereof. Preferably, the DNA molecule comprises a sequence which—when transcribed using a suitable DNA-dependent RNA polymerase—gives the double stranded RNA molecule or a precursor thereof. Thus, the sequences encoding the double stranded RNA molecule or the RNA molecule precursor are preferably operatively linked to suitable expression control sequences.

The strands of the double stranded RNA molecule or the precursor may be chemically and/or enzymatically synthesized, for example, the antisense RNA strand and the sense RNA strands may be synthesized and the strands may be combined to form the double stranded RNA molecule. Alternatively, the precursor of the double stranded RNA molecule may be synthesized and subjected to a processing step, whereby the double stranded RNA molecule is formed. Alternatively, the DNA molecule encoding the double stranded RNA molecule or the precursor thereof may be synthesized and the resulting DNA molecule may be transcribed whereby the double stranded RNA molecule or the precursor thereof is formed and wherein the precursor may be subjected to a processing step whereby the double stranded RNA molecule is formed.

The RNA molecules may contain 3′ overhangs which are stabilized against degradation, e.g. by incorporating deoxyribonucleotides such as dT, and/or at least one modified nucleotide analogue, which may be selected from sugar-, backbone- or nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl andenosine are suitable. In preferred sugar-modified ribonucleotides the 2′ OH-group is replaced by a group selected from H, OR, R, halo, SH, SR, NH₂, NHR, N(R)₂ or CN, wherein R is C₁-C₆-alkyl, alkenyl or alkynyl and halo is F, Cl, Br or I. In preferred backbone-modified ribonucleotides the phosphoester group connecting to adjacent ribonucleotides is replaced by a modified group, e.g. of phosphothioate group. It should be noted that the above modifications may be combined.

Further, the double stranded RNA molecule may comprise modifications at the 5′ end or 3′ terminus of at least one strand. These modifications are preferably selected from lipid groups, e.g. cholesterol groups, vitamins, etc.

The double stranded RNA molecule, or the precursor thereof or the DNA molecule encoding the RNA molecule or the precursor, may be used for the regulation of the expression of a target gene in cell, an organism or a cell-free system or to produce a cell, organism or cell-free system comprising a double stranded RNA molecule with free 5′ and 3′ accessible ends. For this purpose, the molecule is introduced into the cell, organism or cell-free system under conditions under which target-specific nucleic acid silencing occurs with a RISC.

The cell is preferably a eukaryotic cell, more preferably an animal cell, still more preferably a mammalian cell such as a human cell. The organism is preferably a eukaryotic organism, e.g. a mammal including a human. The cell-free system is preferably an extract or a fractionated extract from a eukaryotic cell, e.g. a mammalian cell such as a human cell.

The target gene may be a reporter gene, a pathogen-associated gene, e.g. a viral, protozoal or bacterial gene, or an endogenous gene, e.g. an endogenous mammalian, particularly human gene. The endogenous gene may be associated with a disorder, particularly with a hyperproliferative disorder, e.g. cancer, or with a metabolic disorder, e.g. a disorder associated with carbohydrate, energy, lipid, nucleotide, or amino acid metabolism or a disorder associated with the biosynthesis or metabolism of glycans, polyketides and nonribosomal peptides, cofactors and vitamins or secondary metabolites, with the biodegradation of xenobiotics or with a neurodegenerative disorder such as Alzheimer, Parkinson, Huntington, ALS, MS etc. Thus, the present invention is suitable for the manufacture of reagents, diagnostics and therapeutics.

For pharmaceutical applications, the invention provides also a pharmaceutical composition comprising as an active agent at least one double stranded RNA molecule as described herein, or a precursor thereof or a DNA molecule encoding the double stranded RNA molecule or the precursor and a pharmaceutical carrier. The composition may be used for diagnostic and therapeutic applications in human medicine or in veterinary medicine.

For diagnostic or therapeutic applications the composition may be in form of a solution, e.g. an injectible solution, a cream, ointment, tablet, suspension or the like. The composition may be administered in any suitable way, e.g. by injection, by oral, topical, nasal, rectal application etc. The carrier may be any suitable pharmaceutical carrier. Preferably, a carrier is used of increasing the efficacy of RNA molecules to enter the target cells. Suitable examples of such carriers are liposomes, particularly cationic liposomes.

The double stranded RNA molecules with accessible 5′- and 3′-ends are also suitable for the modulating of a target gene specific silencing activity in a cell, an organism or a cell-free system, wherein the activity of at least one polypeptide of the gene silencing machinery is selectively modulated, e.g. increased and/or suppressed. These polypeptides are preferably selected from Argonaute proteins such as Ago-1, Ago-2, Ago-3 and Ago-4, such as Ago-1 (eIF2C1), Ago-2 (eIF2C2), Ago3 (eIF2C3), Ago4 (eIF2C4), PIWIL 1 (HIWI), PIWIL 2 (HFLI), PIWIL 3 and PIWIL 4 (HIWI 2), more preferably Ago-1 and/or Ago-2, and other proteins of the gene silencing machinery such as Dicer proteins, e.g. Dcr1, or Dcr2; Drosha (Pasha, DGCR8), R2D2 (dsRBD), NR, Fmr1/Fxr, Vig, Tsn, Dmp68, Gemin3, Gemin4, Exportin-5 and Loquacious. Preferably, the polypeptide is an Argonaute or Dicer protein such as Ago-1, Ago2, Dcr1 or Dcr2. In this context, reference is made to the publications Sasaki et al., (Genomics 82 (2003), 323-330) and Sontheimer (Nat. Ref. Mol. Cell. Biol. (2002), 127-38) which are herein incorporated by reference. These publications contain a detailed description of polypeptides of the gene silencing machinery and complexes containing these polypeptides.

By means of this selective activity increase and/or suppression, the efficacy of target-gene specific silencing may be considerably increased. Thus, administration of double stranded molecules directed to the mRNA of a target gene, organism or a cell-free system (as indicated above) may be more effective.

In one embodiment, the activity of at least one polypeptide of the gene silencing machinery is selectively increased. This embodiment preferably relates to a selective increase in the activity of Ago-2. The activity increase may be accomplished for example by overexpression of the polypeptide, e.g. in a target cell or a target organism and/or by adding an excess of the polypeptide, e.g. to a cell-free system. A selective activity increase of Ago-2, for example, leads to a significant increase of gene silencing activity.

In a further embodiment, the activity of at least one polypeptide of the gene silencing machinery is selectively suppressed. This suppression may be accomplished by gene-specific silencing of the polypeptide in the target cell, organism or cell-free system. The gene-specific silencing may comprise, for example, administering double stranded RNA molecules, e.g. siRNA molecules or miRNA molecules, precursors thereof or DNA molecules encoding said RNA molecules or precursors thereof directed to the mRNA encoding the at least one polypeptide of the gene silencing machinery which is to be suppressed. This embodiment particularly relates to a suppression of Ago-1 activity which may be accomplished by administering double stranded RNA molecules, precursors thereof or DNA molecules encoding said RNA molecules or precursors thereof directed against Ago-1 mRNA. Preferably, the RNA molecules directed against Ago-1 mRNA are selected such that they specifically interact with an Ago-1 containing RISC as explained above.

Further, the invention relates to a double stranded RNA molecule with a target gene specific silencing activity which comprises a double stranded portion and optionally at least one 3′ overhang, an antisense RNA strand and a sense RNA strand. The antisense RNA strand is selected to have a sufficient degree of complementarity to the mRNA of a target gene for risk formation, accessible 5′- and 3′-ends which do not form stable intramolecular secondary structures and optionally at least one Wobble base pair between the antisense strand and the target sequence. Further, the invention relates to a precursor of such a double stranded RNA molecule or a DNA molecule encoding the double stranded RNA molecule or the precursor thereof.

The RNA molecule may have a target gene silencing activity of at least 90%, 92%, 94%, 96% or 98% (based on the target gene expression in the absence of the RNA molecule). The gene silencing activity may be determined at concentrations of e.g. 0.001 nM, 0.01 nM, 0.1 nM, 0.5 nM, 1 nM, 5 nM, 10 nM or 50 nM in a suitable test system, e.g. as described in the Examples.

The above molecule is suitable for gene specific silencing of a target gene optionally in combination with other components, e.g. (i) at least one polypeptide of the gene silencing machinery as indicated above or (ii) a nucleic acid encoding this polypeptide, wherein (i) or (ii) is present in an amount or form to provide a selective activity increase of the polypeptide or the nucleic acid.

The composition may be an expression system comprising as component (a) a DNA molecule encoding a double stranded RNA molecule directed to the mRNA of the target gene or a precursor thereof and as component (b) a DNA molecule encoding the polypeptide of the gene silencing machinery wherein DNA molecules (a) and (b) are operatively linked to expression control sequences, either on a single expression vehicle or on a plurality of expression vehicles such as plasmid vectors, viral vectors etc. Alternatively, the composition may be a mixture or kit comprising as component (a) a double stranded RNA molecule directed to the mRNA of the target gene or a precursor thereof and as compound (b) a purified or partially purified polypeptide of the gene silencing machinery or a DNA molecule encoding said polypeptide operatively linked to an expression control sequence. In this embodiment, the polypeptide of the gene silencing machinery is preferably Ago-2 and/or Dicer1 (Dcr1).

Furthermore, the invention provides a composition for target gene specific silencing which comprises a double stranded RNA molecule directed to the mRNA of a target gene, a precursor thereof or a DNA molecule encoding the double stranded RNA molecule or the precursor thereof in combination with (b) a double stranded RNA molecule directed to the mRNA encoding at least one polypeptide of the gene silencing machinery, a precursor of the RNA molecule or a DNA molecule encoding the double stranded RNA molecule or the precursor thereof. More preferably, this composition comprises a combination of (a) a double stranded RNA molecule directed to the mRNA of the target gene and (b) a double stranded RNA molecule directed to the mRNA of a protein of the gene silencing machinery. In this embodiment, the polypeptide of the gene silencing machinery is preferably Ago-1.

The compounds and compositions as described above may be a reagent, e.g. a research tool, a diagnostic or a medicament as described above.

The invention also relates to a cell or non-human organism transformed or transfected with the composition or an expression system comprising the composition which comprises at least one expression vehicle.

The invention also relates to a double stranded RNA molecule with gene silencing activity directed against an mRNA of a polypeptide of the gene silencing machinery as indicated above, e.g. Ago-1, or Ago-2 or the precursor thereof or a DNA molecule encoding said RNA molecule or precursor, wherein the antisense strand has accessible 5′- and 3′-ends and optionally at least one Wobble base pair between antisense strand and target sequence.

The double stranded RNA molecule is preferably chosen such it selectively interacts with a RISC containing the predetermined species of protein, e.g. Argonaute protein. For example, an Ago-2 selective double stranded RNA molecule, e.g. a perfectly base paired double stranded RNA molecule may be used to suppress silencing activity associated with Ago-2 containing RISC. Especially preferred is the use of Ago-1 selective double stranded RNA molecules, wherein the antisense strand and the sense strand comprise at least one mismatch within the double stranded portion of the RNA molecule, for selective inhibition of gene silencing activity associated with Ago-1 containing RISC. The above compounds are suitable for use as a reagent, a diagnostic or a medicament.

Particularly preferred examples of such compounds are as follows:

1. Ago-2-dependent (perfectly base-paired) Ago-1-directed siRNA:

| | |
|---|---|
| Sense: | 5′-UGUAUGAUGGAAAGAAGAAdTdT-3′ |
| Antisense: | 5′-UUCUUCUUUCCAUCAUACAdCdA-3′ |

2. Ago-2-dependent (perfectly base-paired) Ago-2-directed siRNA:

| | |
|---|---|
| Sense: | 5′-GGAGAGUUAACAGGGAAAUdTdT-3′ |
| Antisense: | 5′-AUUCCCUGUUAACUCUCCdTdC-3′ |

3. Ago-1-dependent (imperfectly base-paired) Ago-1-directed SiRNA:

| | |
|---|---|
| Sense: | 5′-UGUACGAUGGAAAGAAGACdTdT-3′ |
| Antisense: | 5′-UUCUUCUUUCCAUCAUACAdCdA-3′ |

In another aspect, the invention features methods for identifying (complementary) nucleic acids and/or their structures which specifically and selectively target mRNA or other RNA molecules and act as antagonist. This invention features the nucleic acids and/or structures of the preferred antagonists if they are derived by other methods than the preferred method of this invention, e.g. by genetic algorithms and/or neuronal nets. Preferred antagonists identified using the method of this invention act as siRNA or miRNA, antisense siRNA or miRNA, antisense nucleic, ribozyme or aptamer in one or more in vitro, ex vivo or in vivo biological assays for detection or destruction of the target RNA molecule. Preferred antagonists identified using the method of this invention act as inhibitors of gene expression or hybridization probes in one or more in vitro, ex vivo or in vivo biological assays. Preferred embodiments of the invention are described in the claims.

The methods of this aspect of the present invention entail identification and design of molecules having particular structures. The methods rely on the use of algorithms for the folding of RNA secondary structures and on experimental data derived from in vitro, ex vivo or in vivo biological assays using the preferred antagonists of this invention and/or mRNAs. These theoretical and experimental data permit the reliable identification of inhibitory siRNA or miRNA, antisense siRNA or miRNA, antisense RNA, antisense oligodeoxyribonucleotides (asODN) and mRNA structures accessible for the preferred antagonists of the present invention. Moreover, the methods of the present invention may be used to characterize, select, and design any other RNA structure of interest. Most importantly these methods can be used to predict best siRNAs or antisense siRNAs with highest rate of success compared to alternative strategies.

The concepts “siRNA”, “miRNA”, “antisense nucleic acid”, and “ribozyme” mean native, semi-synthetic, synthetic, or modified nucleic acid molecules of ribonucleotides and/or deoxyribonucleotides and/or modified nucleotides.

A siRNA or miRNA is a double stranded RNA molecule having a double stranded portion of preferably 9-35 nucleotides and optionally at least one 3′-overhang. The double stranded RNA molecule comprises an antisense strand which has sufficient complementarity to a target RNA for mediating silencing of the target RNA in an RISC. Further, the molecule comprises a sense strand which has sufficient complementarity to the antisense strand to form a double strand which is capable of RISC formation. The length of the sense and the antisense strand are preferably 9-40 nucleotides, more preferably 15-30 nucleotides and most preferably 19-25 nucleotides. The 3′ overhangs preferably have a length of 1-10, more preferably of 1-5, e.g. 1, 2, 3, 4, 5 nucleotides.

The invention encompasses siRNA or miRNA molecules as well as precursors thereof and DNA molecules encoding the RNA molecule or the precursor thereof. Precursors of siRNA molecules are preferably small hairpin (sh) RNA molecule or long double stranded RNA molecules which are processed in a cell to give siRNA molecules. Precursors of miRNAs are preferably primary (pri) miRNA molecules or precursor (pre) miRNA molecules. The DNA molecule encoding the RNA molecule or the precursor thereof is preferably an expression vector, e.g. a plasmid or a viral vector comprising expression control sequences operatively linked to the coding sequences which is capable of expressing RNA molecules or precursors thereof.

The siRNA or miRNA molecules and other RNA molecules may comprise at least one modified nucleotide analogue, e.g. a sugar-, backbone-, or nucleobase-modified analogue as known in the art. Further, double stranded RNA molecules may comprise stabilized 3′ overhangs, which contain deoxyribonucleotides, e.g. dT.

The RNA molecule may further comprise 5′ and/or 3′ modifications, preferably selected from lipid groups, e.g. cholesterol groups and vitamins.

The RNA molecule may be chemically or enzymatically synthesized according to methods known in the prior art. The RNA molecule may be used for regulating the expression of a target gene in a cell, an organism or a cell-free system or for producing a knockdown cell, organism or cell-free system or for examining the function of the target gene in a cell, an organism or a cell-free system.

The concept “target RNA” means a mRNA, pre-mRNA or any other transcript of cellular or non-cellular (i.e. viral, bacterial) origin.

In a preferred embodiment of the present invention the siRNA or miRNA duplex contains an antisense siRNA that folds and/or is predicted to fold no RNA secondary structure or secondary structures comprising free or pseudo-free nucleotides at the 3′ and 5′ end of the molecule.

In another preferred embodiment of the present invention the structure of the mRNA target is accessible to the preferred antagonists of this invention. Accessible sites within the mRNA imply regions of unpaired nucleotides such as loops, bulges, free ends, junctions, and joints, and are defined by the method of this invention.

A further object of the present invention is a vector which contains the above defined siRNA or antisense siRNA (nucleic acid or ribozyme) according to the invention or which contains a corresponding DNA sequence complementary to the antisense nucleic acid which following transcription in suitable host cells results in the above defined siRNA or antisense siRNA according to the invention. The vector according to the invention can preferably contain suitable regulatory elements such as promoters, enhancers, and termination signals. In an embodiment according to the invention the, the vector can be used, for example, for stable integration of the nucleic acid according to the invention into the genetic material of a host cell.

A further object of the present invention is a host cell which contains the siRNA or antisense siRNA or the vector according to the invention. Suitable host cells are, for example, all eukaryotic and prokaryotic cells, preferably human and mammalian cells which carry corresponding target sequences.

A further object of the present invention is an organism which contains the siRNA or antisense siRNA or the vector or the host cell according to the invention. Suitable organisms are, for example, all eukaryotes and prokaryotes, preferably humans and mammals and prokaryotes.

A further object of the invention is a reagent, a diagnostic or a medication which contains the siRNA or antisense siRNA or the vector according to the invention. A medication may possibly contain the molecule in a pharmaceutically acceptable base and/or diluting agent. The medication according to the invention can be used to inhibit or eliminate disease conditions caused by the target sequences through transient or stable integration of the siRNA or antisense siRNA or other antagonist according to the invention by transformation and/or transfection and/or transduction in host cells and/or organisms according to the invention.

A further objective of the invention is a carrier and/or chip which contains nucleic acids according to the invention which can be used to identify and/or detect and/or discriminate target molecules according to the invention for scientific and/or diagnostic purposes.

The methods of the invention for identifying nucleic acids and/or their structures employ computer-based methods for identifying compounds having a desired predictable structure. More specifically, the methods of this invention use computable information on RNA secondary structures of functional RNA molecules in order to predict new improved RNA structures/molecules based on natural and/or artificial sequences. The methods are in silico selection methods, that is, for any target sequence or sequence context of interest complete sequence spaces of potential antagonists or other functional molecules are generated or identical or improved models or signatures of structures which have been proven to show biological activity in a certain sequence context or against a certain target RNA are being selected from structure spaces related to other sequences or mRNAs.

The compounds selected by the methods of this invention show highest biological activity and are biologically active with highest probability of success compared to compounds designed by state of the art methods. The actual activity can be finally determined only by measuring the activity of the compound in relevant biological assays. However, the methods of this invention are extremely valuable because they help to dramatically reduce the number of compounds which have to be tested to identify biologically active molecules.

In general, nucleic acids identified or designed using the methods of this invention can be synthesized chemically or enzymatically or can be transcribed endogenously within target cells and then tested for biological activity using in vitro, ex vivo or in vivo biological assays.

RNA secondary structures may be identified by determining the minimum free Gibbs energy of a given structure and/or by determining a partition function for a given structure. Programs suitable for generating predicted RNA secondary structures from RNA sequences include: mfold versions 2.0 to 3.1 (M. Zuker), RNAfold (P. Schuster) and the McCaskill partition function. In this context, reference is made to Zuker and Stiegler (Nucleic Acids Res. 9, (1981), 133-148) and McCaskill (Biopolymers 29, (1990), 1105-1119), which are herein incorporated by reference.

The methods of the present invention for identifying nucleic acids and/or their structures may be implemented in hardware or software, or a combination of both. They may be implemented in computer programs executing on programmable computers each comprising a processor, a data storage system (including volatile and non-volatile memory and/or storage elements), at least on input device, and at least one output device. Program code is applied to input data to perform the functions described above and below and generate output information. The output information is applied to one or more output devices, in known fashion. The computer may be, for example, a personal computer, microcomputer, workstation, cluster or mainframe of conventional design or arrangement of those.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM, ZIP, or magnetic diskette) readable by a general or special purpose programmable computer, for configuring and operating the computer when the storage media or device is read by the computer to perform the procedures described herein. The inventive systems may also be considered to be implemented as a computer-readable storage medium, where the storage medium so configured causes a computer to operate in a specific and preferred manner to perform the functions described herein. The data sets of the molecular structures invented may also be included in a computer-readable memory and may be administrated in databases.

The programs of this invention are parallelizable, that is, using parallelized computers or processors, the desired functions may be processed in parallel on all available CPUs, thus strongly reducing the relative processing times. Due to their parallelizability the invented programs are suitable to master high quantities of data and are thus compatible with experimental high throughput technologies (high-throughput compatible).

1) Design of RNA Antagonists of mRNA Structures

FIG. 17 shows the flowchart of method 1 for the selection of RNA antagonists of mRNA structures using a computer system. The method uses a programmed computer comprising a processor, a data storage system, at least one input device, and at least one output device, and comprises the steps of:
(1) inputting into the programmed computer through an input device data comprising the primary structure (sequence) of L nucleotides (nt) in length of a target sequence (mRNA, cDNA, gene) either manually, by copying or by fetching from a database (STEP 100).
(2) generating, using the processor, the reverse complement (antisense sequence) of the input sequence (STEP 102).
(3) generating, using the processor, a sequence space having a plurality of sequence space members, e.g. the complete set of subsequences of a defined length l contained in the reverse complement of the input sequence (STEP 104). The set of subsequences is termed antisense sequence space of the target sequence and has a diversity of L-I different sequences. I may be freely chosen. For the selection of siRNAs or antisense siRNAs I is typically 19 nt but may also be longer or shorter (FIG. 21). For the selection of antisense RNAs I may be chosen between 50 and several hundred nucleotides in length (FIG. 23). If desired, the sequence space may be expanded,
(4) Optionally, generating, using the processor, new sequences by base exchanges within members of sequence spaces as depicted in FIG. 20. Preferably one or more cytosine (C) is exchanged by Uracil (U) and one or more Adenine (A) is exchanged by Guanine (G) thereby expanding the diversity of sequences spaces, further generating Wobble (G-U, U-G) base pairs. (STEP 105).
(5) optionally, fusing, using the processor, constant sequences to the ends of all members of the sequence space and/or inserting, using the processor, constant sequences into defined positions of all members of the sequence space (STEP 107).
(6) Eliminate, using the processor, members of the sequence space which contain defined undesirable sequence motifs such as G or C quartets and/or immunostimulatory motifs (STEP 108).
(7) folding, using the processor and a program suitable for folding RNA secondary structures (such as those described above or equivalents), the complete set of RNA secondary structures, the antisense RNA structure space, corresponding to each of the sequences of the antisense sequence space (STEP 110).
(8) calculating, using the processor, a set of RNA structure parameters, including length of free ends, number and size of joints, number of components, number and size of stem loops and internal loops, and number of junctions (FIG. 25), for each member of the sequence space (STEP 112). The set of parameters of each structure represents a structural signature. Structural signatures may be but must not be unique for a given structure space.
(9) comparing, using the processor, the sets of parameters of the structures of the structure space to a criteria set of parameters representative for inhibitory siRNA or antisense siRNA or antisense RNA respectively (STEP 114).
(10) selecting from the calculated parameter sets, using the processor, structures with sets of parameters corresponding to those of inhibitory siRNAs or antisense siRNAs or antisense RNAs or ribozymes (STEP 116). Criteria sets of inhibitory siRNAs or antisense siRNAs comprise unstructured sequences, and structures with free or accessible ends (FIG. 10), criteria sets of inhibitory antisense RNAs comprise unstructured sequences and structures with free ends and/or stretches of more than ten contiguous unpaired nucleotides in loops, bulges or joints, and/or structures composed of several structural components.
(11) compare, using the processor and a program suitable for comparing sequences with sequences in databases, the corresponding target sites of selected sequences/structures to the appropriate genome database (human, mouse, rat, etc.) and eliminate from consideration any target sequences with more than 15-17 contiguous base pairs of homology to other coding sequences (STEP 118).
(12) ranking, using the processor, remaining candidate sequences according to structural criteria and/or GC content and/or homology to other sequences and/or position within the target sequence (STEP 120).
(13) outputting to an output device the selected and ranked sequences (STEP 122).
   2) Selection of Accessible Sites of mRNA Structures

FIG. 19 shows the flowchart of method 2 for the identification of sites of mRNA sequences/structures accessible for the above defined RNA antagonists using a computer system. The method uses a programmed computer comprising a processor, a data storage system, at least one input device, and at least one output device, and comprises the steps of:
(1) inputting into the programmed computer through an input device data comprising the primary structure (sequence) of L nucleotides (nt) in length of a target sequence (mRNA, cDNA, gene) either manually, by copying or by fetching from a database (STEP 100).
(2) generating, using the processor, a subset of sequences (windows) of a defined length l of the input sequence which are partly overlapping and shifted relative to each other by a shift wide s (STEP 102). The length l of the windows is typically 1000-1400 nt and s should be 1 to 500 nt. Target sequences shorter than 1400 nt in length are not subdivided into shorter windows (FIG. 24).
(3) folding, using the processor and a program suitable for folding RNA secondary structures (such as those described above or equivalents), the minimum free energy structure and a set of higher free energy structures of each sequence window of the target sequence (STEP 104). Typically 5 to 10 higher free energy structures are folded which differ in the basepairing of 20 nt for l=1400 nt.
(4) alignment, using the processor, of all information on Watson-Crick base pairing of all minimum free energy and higher free energy structures of each overlapping window with respect to the nucleotides of the target sequence (STEP 106). The aligned information is: (i) baise-pairing (yes or no) and (ii) kind of open structural element the nucleotide belongs to in case nucleotide is predicted to be not involved in base pairing.
(5) calculating, using the processor, the probability ρ(n) of each nucleotide n of the target sequence of being unpaired. In order to calculate these probabilities, the impact of information derived from different free energy structures is weighted on the basis of empirical rules. Highest impact (factor 20) is assigned to the lowest free energy structure (s1) followed by impact factors 10, 5, 2, and 1 for the 2^{nd}, 3^{rd}, 4^{th}, and 5^{th} lowest free energy structures (s2 to s5). These impact factors are examples and more than five low free energy structures may be considered. The information unpaired (1) or paired (0) of a single nucleotide within a structure s1 to s5 is multiplied with the corresponding impact factor, summed up, and divided by the maximum value (by 38=20+10+5+2+1 in this example) (STEP 108).
(6) Identifying, using the processor, accessible target sites. Accessible target sites are characterized by contiguous stretches of more than 7, preferably more than 10, unpaired nucleotides (STEP 110). Unpaired nucleotides are preferably contained in free ends, joints, loops, and bulges (FIG. 25).
(7) selecting from the calculated parameter sets, using the processor, structures with sets of parameters and positions corresponding to those of inhibitory siRNAs or antisense siRNAs or antisense RNAs as determined by the first method of this invention method (STEP 112). Target sites of antisense oligodeoxyribonucleotides (asODN) (FIG. 22) are selected according to the calculated accessibility score and in a way that the 5′ and/or the 3′ end, preferably the 3′ end, of the asODN overlaps with accessible target site.
(8) compare, using the processor and a program suitable for comparing sequences with sequences in databases, the corresponding target sites of selected sequences/structures to the appropriate genome database (human, mouse, rat, etc.) and eliminate from consideration any target sequences with more than 15-17 contiguous base pairs of homology to other coding sequences (STEP 114).
(9) ranking, using the processor, remaining candidate sequences according to structural criteria and/or GC content and/or homology to other sequences and/or position within the target sequence (STEP 116).
(10) outputting to an output device the selected and ranked sequences (STEP 118).

Throughout this description, the preferred embodiments and examples shown should be considered as exemplars, rather than as limitations on the present invention.

Further, the present invention shall be explained in more detail by the following figures and examples.

### DESCRIPTION OF DRAWINGS

FIG. 1: In silico-selected jagged-1-directed guide-siRNA. a, Jagged-1 target sequence and overlapping as-siRNA sequences. The common core sequence is shaded in grey. Nts forming a conserved tetra-loop are framed. b, Predicted secondary structures of as-siRNA, structural signatures, calculated target accessibilities, and siRNA activities. c, Jagged-1 expression (MFI) in cells transfected with 1(□) or 10(▪) pmol siRNA duplexes. (a) and (b), Identical sequence stretches are colour-coded. Error bars represent standard deviations (SD) of 3 to 6 experiments.

FIG. 2: Activities of in silico selected siRNA. a, Knock-down of GFP and b Luciferase gene expression. G: GFP-directed siRNA; L: Luciferase-directed siRNA; US: unstable; RC: random-coiled; IL: intern loop; 2SL: 2 stem loops; h/m/l: high/medium/low energy; C: Control siRNA; /: Mock-transfected cells; error bars represent SD of 3 to 6 experiments. Correlations r between free 3′ nts or ΔG of structures in (a) and (b) and gene silencing. *21/2=10.5 nts were assigned to 3′ ends of unstructured RNA; ∘pseudo-free 3′ nts resulting from opening of 3′-adjacent stems divided by 2.

FIG. 3: Programming active as-siRNA/guide-RNA structures by base exchanges. a, A>G and C>U exchanges (red) can program structures and/or ΔG of guide-RNA thereby inducing wobble-base pairing with the target but preserving target complementarity. b, Jagged-1 expression (MFI) in 293T cells transfected with siRNA duplexes containing parental and programmed as-siRNA strands. Duplexes form only Watson-Crick bp. *Partition structures. RC: Randomly-coiled as-siRNA. Error bars represent SD of 3-4 measurements. c, Dimensions of guide-siRNA sequence spaces without (D₁) and including base exchanges (D₂) for a given target RNA of L nts in length containing guanine (G) and uracil (U) bases.

FIG. 4: SiRNA duplex structures determine Argonaute dependence of RISC. Jagged-1 expression (MFI) in Ago-1 (▪), Ago-2 (□), and Ago-1+2 (▪) knock-down or native (□) 293T cells. D-wob, duplex-intrinsic base wobbling (blue); t-wob: target base wobbling (red); d-mis, duplex-intrinsic mismatching. *Partition structures. Error bars represent SD of 3-4 measurements.

FIG. 5: Structures of guide-siRNA are correlated with RNA i. A, Mfe structures of guide siRNA corresponding to each 13 active and inactive published siRNA^{9,11,13,30-32} targeting 5 mRNA targets were predicted. Predictions based on the canonical AUCG base alphabet and for consistency with physical structures were preferably considered siRNA with XY or dXdY 3′-overhangs for analysis. Structures were characterized by numbers of terminal free nts, loop size, bp, and ΔG of secondary structure formation. Error bars represent averages or numbers of structures; maxima and minima are indicated. b, consensus structures derived from active and inactive guide-si RNA species.

FIG. 6: RNAse T1 probing of guide-siRNA structures 4-7, 0-0, and 2-9. Guide-siRNA strands were 5′-labeled with ³²P using polynucleotide kinase (MBI Fermentas, St. Leon-Rot, Germany) and [γ³²P]-ATP. Labeled RNA was denatured at 90° C. for 2 min, slowly cooled down to room temperature to allow for intramolecular structure formation, and exposed for 15 min at room temperature to 0.1, 0.001, and 0.001 U/μl Rnase T1 (Ambion, Austin, USA) respectively. Rnase T1 specifically cleaves single stranded RNA after guanosine residues. Cleavage products were separated by denaturing 15% PAGE prior to autoradiography of dried gels. Predicted mfe structures, cleavage sites, and sites protected upon base pairing are indicate. [symbol] : strong cleavage, [symbol] : weak cleavage, (G): protected G. C: Control lanes.

FIG. 7: Prediction and proof of target structure accessibilities. Predicted mfe structures and accessibility profiles of local mRNA targets a, T, b, T-a, and c, T-i. Bases targeted by siRNA or asODN are indicated at the structures. Accessibility profiles, representing accessibility probabilities of individual bases derived from the complete Boltzmann ensemble of secondary structures were calculated using the program TARGETscout. The accessible loop structure L1 in (b) is highlighted in light blue. d, Jagged-1 expression (MFI) in cells transfected with 100 (▪) or 500 (□) pmol asODN corresponding in sequence to guide-siRNA targeting T, T-a, and T-i. Error bars represent standard deviations of 3 experiments.

FIG. 8: Classifying guide-RNA structures. a, Classification of guide-structures according to accessibility of 5′/3′ ends and ΔG. Random coils (RC are most active, followed by stem-loop structures with free 5′ and 3′ nts (X-Y) and internal loop (IL) or 2-stem-loop (2SL) structures with pseudo-accessible ends. Structures lacking free 5′ and/or 3′ nts (0-X, 0-0, X-0) are inactive. Unstable structures (US) can fall into potential holes of active or inactive structures according to ambient conditions. b, Probability P of structure formation in dependency of ΔG. Considering 2 states, mfe folding and RC, then P is given by exp(−ΔG/RT)/(1+exp(ΔG/RT)). R: Universal gas constant; T: Absolute temperature.

FIG. 9: Model describing the determination of RNA silencing by RNA secondary structures. SiRNA duplexes are recognized, unwound, and guide-strands are incorporated into RISC. Perfectly matching duplexes induce formation of Ago-2-containing RISC, mismatching duplexes induce formation of Ago-1-dependend complexes. Guide-strands linked to RISC can form stable secondary structures. Guide-RNA structures determine strength of silencing correlating with accessibility of terminal nts increasing form complexes I to VII. mRNA-targeting initiates via free ends of guide-structures, base-matching with guide-RNA 5′domains monitors for target specificity. Upon targeting, wobble pairings with guide-RNA 5′ regions induce reprogramming or resolving of RISC* leading to Ago-1/2-independent silencing or antisense effects.

FIG. 10: Programming Argonaut-dependence of RISC by siRNA structure design. Wobble base pairing between target and the 5′terminus of the guide-strands prevents Ago-1 and Ago-2 dependency (1). Conventional duplexes (2) and those inducing target wobbling through central regions of the guide strand (3,4,5) mediate Ago-2-dependent gene silencing. Mismatches within siRNA duplexes (6,7) result in Ago-1-dependent silencing. Inhibition of jagged-1 gene expression (MFI) 72 h post transfection of 293T cells with jagged-1-specific siRNA (Control) or cotransfection including Ago-1, Ago-2, or Ago-1+2-specific siRNA. SD of 3 independent measurements.

FIG. 11: Co-delivery of Ago-1-specific siRNA enhances gene knock down mediated by target-specific siRNA, the activity of which depends on Ago-2 protein. Inhibition of jagged-1 gene expression 72 h post transfection of 293T cells with jagged-1-specific siRNA or co-transfection including Ago-1-specific siRNA. SD of 3 independent measurements.

FIG. 12: Co-delivery of Argonaute-specific siRNA can modulate knock down of the Luciferase gene expression mediated by 10 pmol/well Luciferase-specific siRNAs (siRNA-1 and siRNA-2). Silencing activity decreases with the delivery of Ago-2-dependend (perfectly base-paired duplex) Ago-2-specific siRNA (si-Ago-2). Silencing activity is increased if only 5 pmol/well Luciferase-specific siRNA is delivered together with 5 or 0.5 pmol/well of a Ago-2-dependend (perfectly base-paired duplex) Ago-1-specific siRNA si-Ago-1-2). An Ago-1-dependent (imperfectly base paired duplex) Ago-1-directed siRNA (si-Ago-1-1) which does not compete for cellular Ago-2 protein supports knock down most efficiently. Luciferase gene expression was detected 48 h post transfection of 293T cells with a Luciferase gene expression vector (pGL2) and different Luciferase- and Ago-specific siRNAs. SD of 3 independent measurements.

FIG. 13: Over-expression of Argonaute-2 protein enhances specific siRNA-mediated knock-down of Luciferase gene expression. Gene expression was monitored 48 h post transfection of 293T cells with 500 ng/24-well pGL2 Luciferase expression vector. a, Cell were additionally transfected with 500 ng/24-well Argonaute-2 expression vector (pAgo-2) and/or 2.5 pmol/24-well Luciferase-specific siRNA (Luci-siRNA). b, analog to a but total amounts per well of plasmid DNA and siRNA were adjusted using control DNA (pControl) and RNA (Control-siRNA). SD of 3 independent measurements.

FIG. 14: Replication of S. thyphimurium (CFU) 5 h post infection in control (transfected with control siRNA) and RNAi-disabled (RNAi KO) HEK293T cells transfected with Ago-1+Ago-2-directed siRNAs.

FIG. 15: Replication of GFP-expressing S. thyphimurium (a, GFP expression; c, CFU) and L. monocytogenes (b, GFP expression; d, CFU) in control cells compared to Ago-1, Ago-2, and Dicer1 knock-down HEK293T cells 6 h post infection.

FIG. 16: Transfection of HEK293T cells with Argonaute-2 expressing plasmid DNA decreases susceptibility to S. thyphimurium. a, Bacterial growth (cfu) in infected cells. b and c, Bacterial proliferation in infected cells monitored by bacterial EGFP expression 12 and 36 h post infection.

FIG. 17: is a flowchart showing a second method for identifying sites of mRNA molecules accessible for antagonists using a computer algorithm.

FIG. 18: schematically shows how base exchanges expand sequence and structure spaces of mRNA antagonists, i.e. antisense siRNAs.

FIG. 19: is a flowchart showing a second method for identifying sites of mRNA molecules accessible for antagonists using a computer algorithm.

FIG. 20: is a schematic representation of structures of mRNA antagonists, i.e. antisense siRNAs, which can be selected by the first method of this invention.

FIG. 21: is a schematic representation of accessible sites of mRNA structures which can be selected by the second method of this invention.

FIG. 22: is a graphic representation which shows the inhibition of gene expression by siRNA/antisense siRNA molecules selected by the first method of this invention.

FIG. 23: schematically shows a complete antisense RNA (asRNA) sequence space directed against a given target which can be generated by the first method of this invention.

FIG. 24: schematically shows a complete space of local target sites of a target RNA molecule which can be generated by the second method of this invention.

FIG. 25: schematically describes examples of structural elements which are analyzed by the methods of this invention and which represent the basis for the calculation of RNA secondary structure parameters.

FIG. 26: is a schematic representation of structures of mRNA antagonists, i.e. antisense siRNAs, which can be selected by the first method of this invention.

FIG. 27: is a schematic representation of accessible sites of mRNA structures which can be selected by the second method of this invention including loops, bulges, joints, and free ends (A). These structural elements are considered accessible only if they are conserved among all analyzed windows and optimal (minimum free energy) as well as suboptimal foldings (B).

### EXAMPLES

### 1. Methods

SiRNA/asODN preparation and design. SiRNA were selected using the algorithm siRNAscout (STZ Nucleic Acids Design, Berlin, Germany) targeting coding sequences. SiRNA single-strands were synthesized at Xeragon or Dharmacon as 21-mers, sense strands with dTdT 3′-ends antisense strands with dXdY 3′-ends including dT or dU nts (jagged-1) or XY 3′-ends (Luciferase and GFP). SiRNA strands were annealed according to manufacturer's instruction resulting in 19 bp duplexes with 2 nt 3′ overhangs. Qualities and quantities of ssRNA and duplexes were monitored using a bioanalyzer (Agilent Technologies, Palo Alto, USA). Jagged-1-directed siRNA not included in Figures: t-a: sense:

5′-GAAACAGUAGCUGCCUGCCdTdT-3′, antisense:
5′-GGCAGGCAGCUACUGUUUCdGdG-3′; t-I: sense:
5′-ACUUGCAUCGAUGGUGUCAdTdT-3′, antisense:

### 5′-UGACACCAUCGAUGCAAGUdGdC-3′. Luciferase-directed si RNA: L-RC:

sense: 5′-GAGGAGUUGUGUUUGUGGAdTdT-3′, antisense:
5′-UCCACAAACACAACUCCUCCG-3′; L-3-10: sense:
5′-UCGGGGAAGCGGUUGCAAAdTdT-3′, antisense:
5′-UUUGCAACCGCUUCCCCGACU-3′; L-US: sense:
5′-ACGACAAGGAUAUGGGCUCdTdT-3′, antisense:
5′-GAGCCCAUAUCCUUGUCGUAU-3′; L-2SL: sense:
5′-CGUUCGGUUGGCAGAAGCUdTdT-3′, antisense:
5′-AGCUUCUGCCAACCGAACGGA-3′; L-5-6-h: sense:
5′-AAAACGGAUUACCAGGGAUdTdT-3′, antisense:
5′-AUCCCUGGUAAUCCGUUUUAG-3′; L-5-6-m: sense:
5′-AUGUGUCAGAGGACCUAUGdTdT-3′, antisense:
5′-CAUAGGUCCUCUGACACAUAA-3′; L-5-6-I: sense:
5′-AUCUACCUCCCGGUUUUAAdTdT-3′, antisense:
5′-UUAAAACCGGGAGGUAGAUGA-3′; L-IL: sense:
5′-AUUCUGAUUACACCCGAGGdTdT-3′, antisense:
5′-CCUCGGGUGUAAUCAGAAUAG-3′; L-5-0: sense:
5′-AACGCUUCCAUCUUCCAGGdTdT-3′, antisense:
5′-CCUGGAAGAUGGAAGCGUUUU-3′; L-0-0: sense:
5′-UACAUUCUGGAGACAUAGCdTdT-3′, antisense:
5′-GCUAUGUCUCCAGAAUGUAGC-3′. GFP-directed siRNA: G-US1: sense:
5′-AGCGCACCAUCUUCUUCAAdTdT-3′, antisense:
5′-UUGAAGAAGAUGGUGCGCUCC-3′; G-US2: sense:
5′-AACGUCUAUAUCAUGGCCGdTdT-3′, antisense:
5′-CGGCCAUGAUAUAGACGUUGU-3′, G-5-6-T1: sense:
5′-CGGCAUCAAGGUGAACUUCdTdT-3′, antisense:
5′-GAAGUUCACCUUGAUGCCGUU-3′; G-5-6-T2: sense:
5′-AGAAGCGCGAUCACAUGGUdTdT-3′, antisense:
5′-ACCAUGUGAUCGCGCUUCUCG-3′; G-2SL: sense:
5′-GCCCUGGCCCACCCUCGUGdTdT-3′, antisense:
5′-CACGAGGGUGGGCCAGGGCAC-3′; G-IL: sense:
5′-UGGAG UACAACUACAACAGdTdT-3′, antisense:
5′-CUGUUGUAGUUGUACUCCAGC-3′; G-1-0: sense:
5′-ACAACGUCUAUAUCAUGGCdTdT-3′, antisense:
5′-GCCAUGAUAUAGACGUUGUGG-3′. Ago-1/2-specific siRNA were selected with siRNAscout having a minimum of cross-homology to the Ago-2/1 mRNA respectively. Ago-1: sense:
5′-UGUAUGAUGGAAAGAAGAAdTdT-3′, antisense:
5′-UUCUUCUUUCCAUCAUACAdCdA-3′; Ago-2: sense:
5′-GGAGAGUUAACAGGGAAAUdTdT-3′, antisense:
5′-AUUUCCCUGUUAACUCUCCdTdC-3′. AsODN were selected using the algorithm TARGETscout (STZ Nucleic Acids Design, Berlin, Germany) and synthesized at Thermo Electron (Ulm, Germany) with each 2 5′ and 3′ terminal phosphothioate bonds.

Construction and purification of plasmids. A fragment containing the human jagged-1 cDNA (accession no. AF003837) was excised by BamHI and SalI digestion from vector pBabe-Jagged-1 and subsequently cloned into the pcDNA3.1(+) plasmid (Invitrogen, Carlsbad, USA) using the unique BamHI and XhoI restriction sites resulting in jagged-1 expression vector pcDNA-Jagged-1. All plasmids were prepared using the Endofree Plasmid Maxi Kit™ (Qiagen, Hilden, Germany). For RNA co-transfection, plasmid DNA was further purified under RNAse-free conditions by repetitive phenol extraction.

Evaluation of siRNA/asODN activity in tissue culture. GFP positive HEK 293T cells were analyzed for jagged-1 expression 72 h after co-transfection of siRNA (0.1-100 pmol) or asODN (100 or 500 pmol), jagged-1 expression vector pcDNA-Jagged-1, and pEGFP-C1 (BD Biosciences Clontech, Palo Alto, USA) using Lipofectamine 2000 according to manufacturer's instructions (Invitrogen). Cells seeded in 24 well plates were detached using PBS containing 2 mM EDTA and subsequently stained with biotin-conjugated anti-jagged-1 (R&D Systems, Abingdon, UK) and allophycocyanin-conjugated streptavidin (BD Biosciences Pharmingen, San Diego, USA). Jagged-1 expression was analyzed on a FACS Calibur™ (BD Biosciences Immunocytometry Systems, San Jose, USA), and quantified by gating on GFP positive cells and determining the median fluorescence intensity (MFI) of jagged-1 staining. Alternatively, the percentage of jagged-1 positive cells was measured. Apparent values of half maximal inhibition (IC₅₀ values) were determined from MFI values using the program GraFit (Erithacus Software, Horley, UK). To detect Ago-1 and 2 protein dependence of jagged-1 silencing, 293T cells were pre-transfected with 480 pmol Ago-1- and/or 480 pmol Ago-2-siRNA in 75 cm² tissue culture flasks. After 48 h, cells were co-transfected and processed as described above with 20 pmol jagged-1 siRNA additionally including 50 pmol Ago-1- and/or 50 pmol Ago-2-siRNA per 24 well. Expression of Firefly luciferase in 293T cells was analyzed 48 h post co-transfection of 20 pmol siRNA and pGL2-Basic (Promega, San Luis Obispo, USA). Activities of GFP-directed siRNA were monitored in 293T cells by fluoroscan using the Fluorskan Ascent fluorometer (Thermo Labsystems, Helsinki, Finland) 48 h post co-transfection of 20 pmol siRNA and pEGFP-C1.

Thermodynamic duplex profiling. Free energy values representing internal average stabilities of pentamer subsequences within siRNA duplexes were calculated using the program OligoWalk²⁸.

RNA secondary structure prediction. Mfe structures were predicted based on default parameters of mfold2.0 (ref. 19). Partition structures were predicted based on mfold2.0 default parameters implemented into the dynamic programming algorithm of the Vienna RNA package²⁹. For sequences selected in this study, mfe and partition structures are identical except for as-siRNA 2-4, IL1, and IL2. For these sequences partition structures are better compatible with our model.

### 2. Results and Discussion

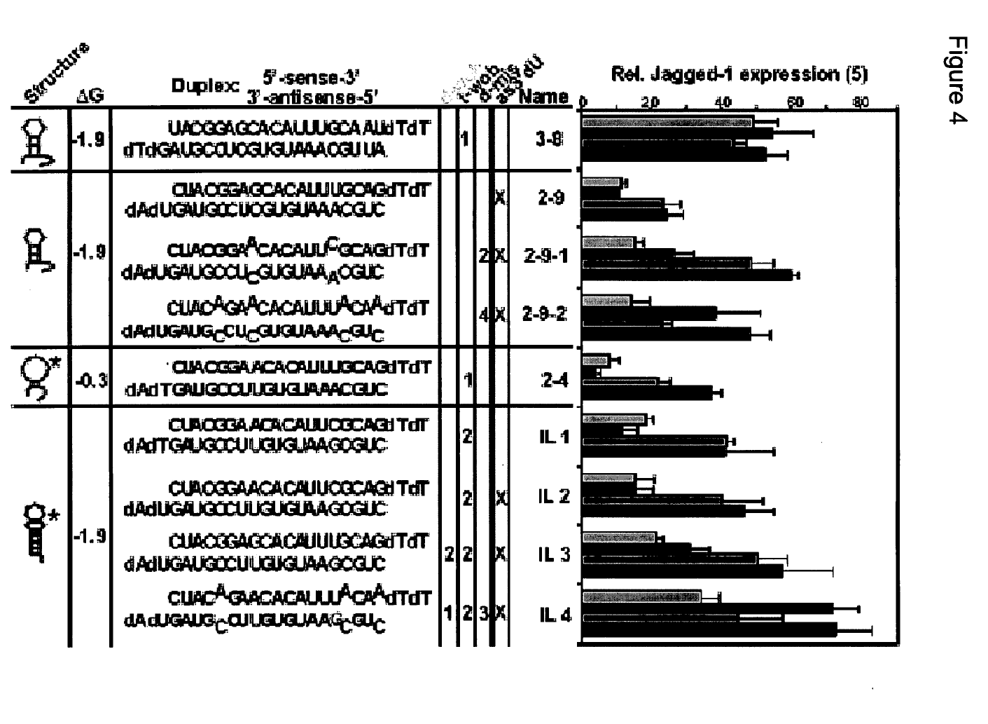

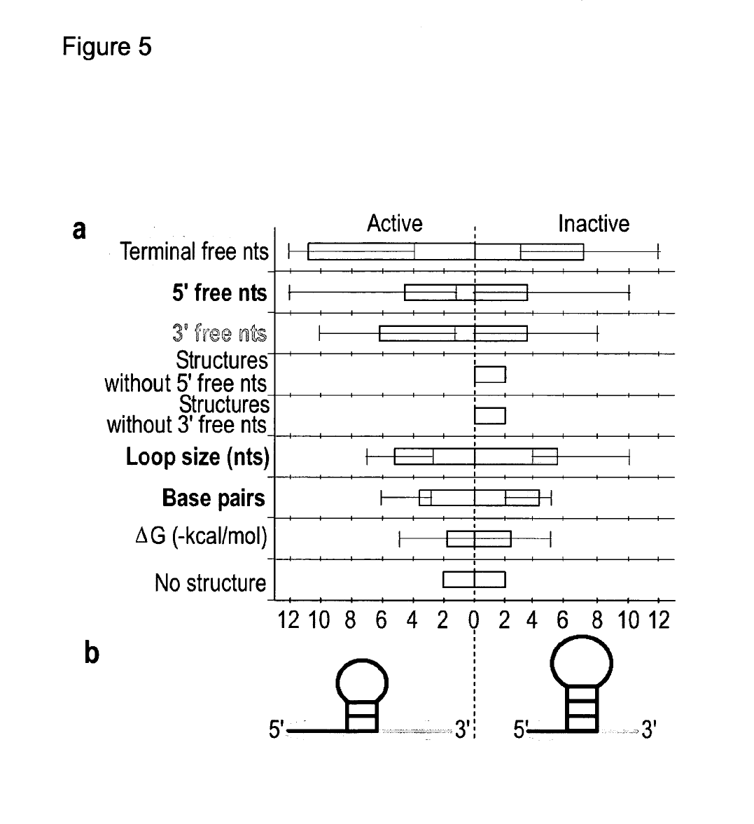

We interrogated the potential role of secondary structures of as-siRNA in RNAi. Secondary structures of as-siRNAs relating to active and inactive siRNA duplexes were predicted using mfold¹⁹ and McCaskill's partition function²⁰. The vast majority (69%) of as-siRNA structures encompass stem-loops with or without single-stranded 5′ and 3′ ends. Active structures contained more terminal free nts, mainly at the 3′ ends, compared to inactive and random structures (see FIG. 5). Structures without free nts at either terminus were only observed among inactive sequences and about 1 in 5 active or inactive as-siRNA failed to form stable structures. We hypothesize that single-stranded ends of as-siRNA structures are required for efficient induction of RNAi.

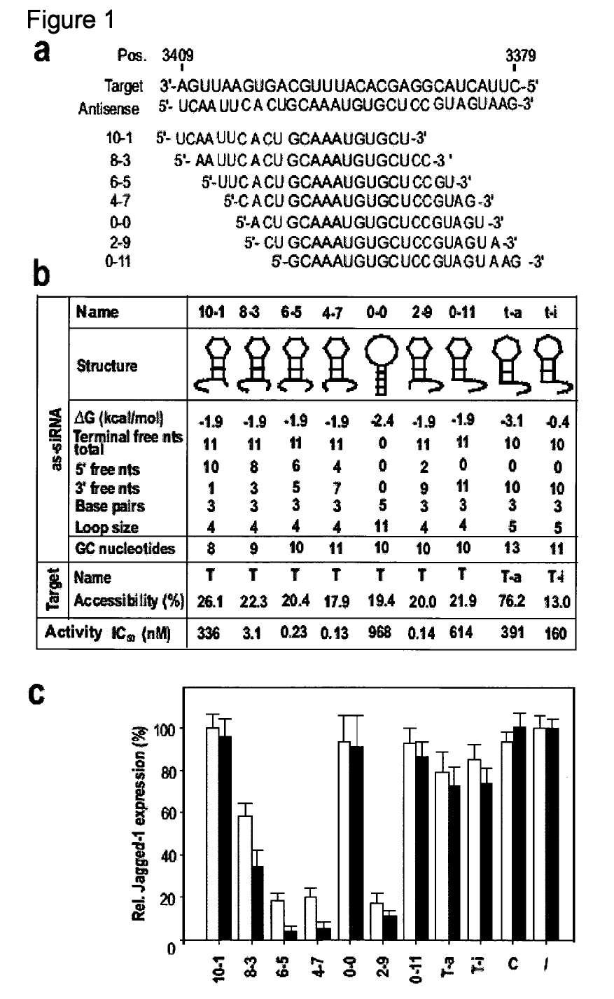

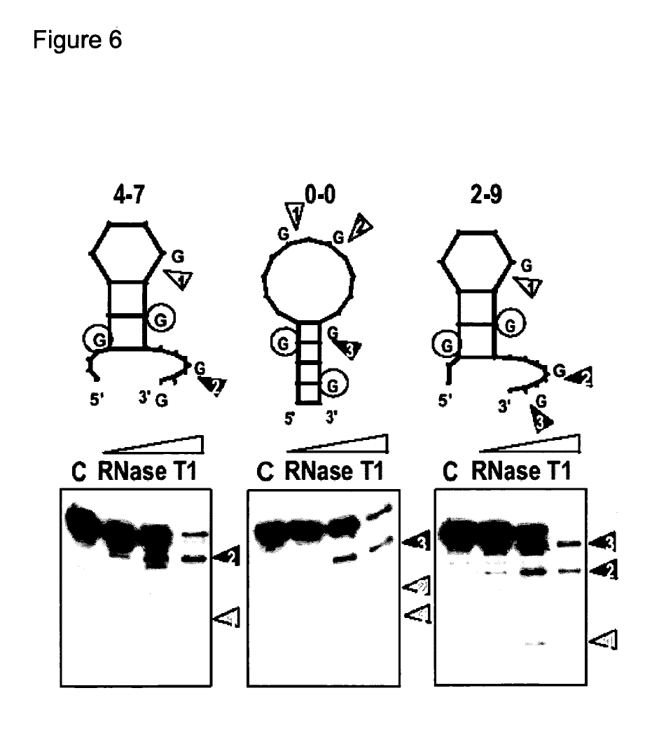

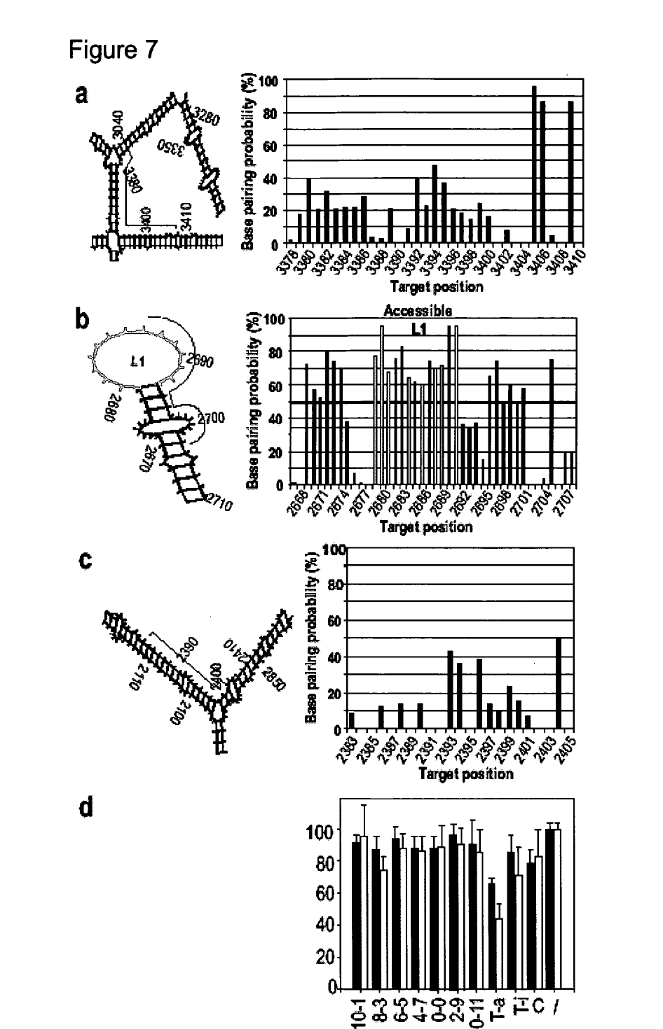

We developed a structure-based siRNA selection program and identified a set of overlapping (1 or 2 nt shifts) as-siRNA sequences directed against the human jagged-1 gene relating to structures containing a conserved stem-loop element and 11 terminal unpaired nucleotides, the latter of which can be assigned either to the 5′ end, to the 3′ end or to both termini at varying distribution (FIG. 1). Selected structures were suitable to systematically evaluate the impact of terminal free nts of as-siRNA structures on RNAi, independent of Gibbs free energies (ΔG) of structure formation and target-related influences. Structures were termed according to numbers of free nts assigned to the termini, e.g. structure 6-5 comprises 6 5′ and 5 3′ unpaired nts. Notably, favorable structures 4-7 and 2-9 were predicted to frame unfavorable structure 0-0 (not a putative structure 3-8) without free nts at any terminus (FIG. 1b). Transitions from structure 4-7 to 0-0 to 2-9 were confirmed by enzymatic RNA secondary structure probing in vitro (see FIG. 6). The local mRNA target region T corresponding to the selected as-siRNAs was predicted inaccessible and unfavorable for targeting by complementary nucleic acids. To investigate target structure roles independently of as-siRNA structures, we selected as-siRNA structures t-a and t-i of the type 0-10, both identical in geometry and unfavorable in terms of silencing but directed against an accessible (T-a) or an inaccessible (T-i) target region (FIG. 1b and FIG. 7).

Activities of duplexes containing the selected as-siRNA strands were tested in human cells in transient assays. Target gene expression was monitored (FIG. 1c) and doses resulting in 50% inhibition (IC₅₀) of jagged-1 expression were calculated. Strongest inhibition (IC₅₀˜0.1 nM) was determined for structures 6-5, 4-7, and 2-9 containing ≧5 free 3′ and ≧2 free 5′ nts (FIG. 1b). Poor effects (IC₅₀˜10² nM) were observed for structures 0-11, t-a, t-i, and 10-1. Structure 0-0 did not show any activity (IC₅₀˜10³ nM). Thus, free 3′ and 5′ ends of as-siRNA structures were critical for RNAi. A single-nt shift from siRNA 4-7 to siRNA 0-0 resulted in 7,000-fold higher IC₅₀ and by a further single-nt shift towards siRNA 2-9, full activity was restored. These differences are independent of target structure but reflect structural changes of as-siRNAs. We found only poor correlations between IC₅₀ values and thermodynamic duplex profiles, base preferences, and low 5′-antisense duplex stabilities, reported previously to correlate with siRNA activity⁸⁻¹². Target accessibilities did not correlate with RNAi either. We observed a correlation between concomitant occurrence of >1 5′ and >3 3′ unpaired nts within stem-loop structures of as-siRNAs and RNAi. For as-siRNA structures containing ≧2 unpaired 5′ nts, numbers of free 3′ nucleotides strongly correlated (correlation coefficient r=0.94) with siRNA activity (1/IC₅₀). Other structural parameters of as-siRNAs did not correlate with RNAi (FIG. 1b).

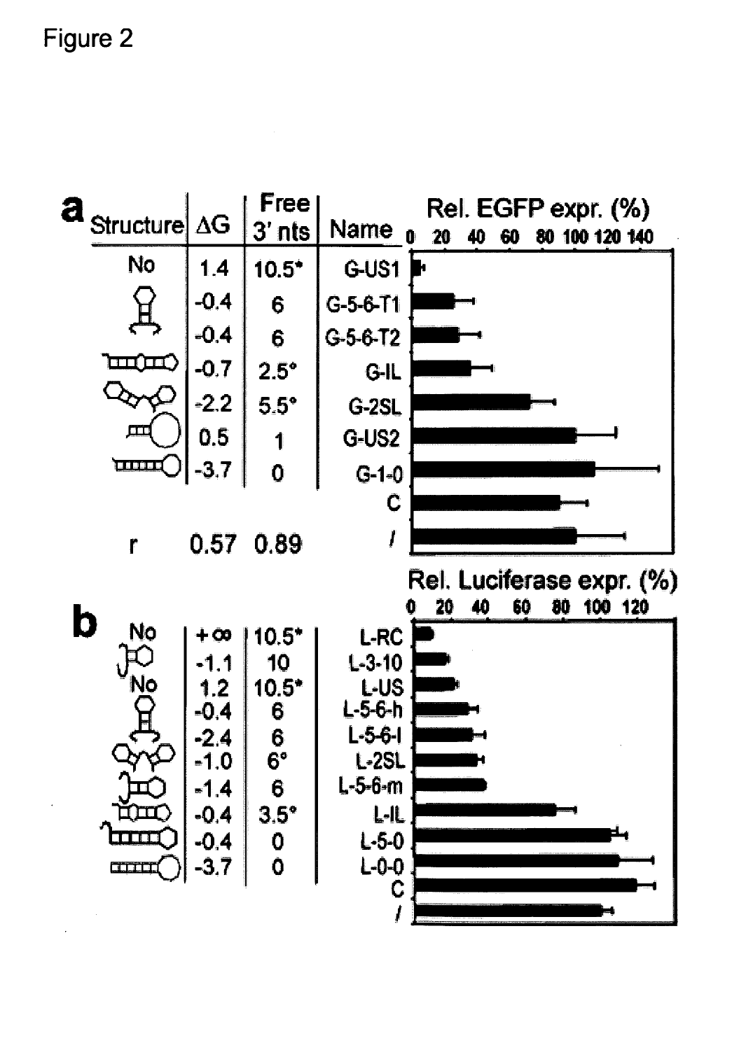

Extrapolating the observed relationship, completely unstructured as-siRNA strands should be most favorable and ΔG could show reciprocal correlation to the silencing activity. Mature miRNAs represent natural counterparts of as-siRNAs and systematic analyses revealed that structures of mature human miRNA²¹ are thermodynamically less stable (higher energy value) compared to structures based on random or human coding sequences (data not shown). Unstable structures (ΔG>0) and RNA which cannot form any stable or unstable secondary structure (unstructured RNA; ΔG=+infinite) due to missing base-pairing possibilities are with 32% more abundant in miRNA compared to random structures (24%). Structures with internal loops (IL) or two stem-loops (2SL) appear more frequently among miRNA. Contrary to statistics, structures without free terminal nts were not observed among miRNA. Thus, termini and/or folding energies of mature miRNA structures appear crucial for miRNA action. We assessed unstructured sequences which can be described by a random coil polymer conformation (RC), unstable structures (US), IL and 2SL structures, and stem-loop structures directed against the mRNAs of the firefly luciferase (L) and the green fluorescent protein (GFP) (FIG. 2). Type 5-6 stem-loop structures identical in geometry but differing in ΔG (L-5-6-h, -m, and -l) or identical in geometry and energy but directed against different target regions (G-5-6-T1 and -T2) showed similar activities indicating that shapes of structures and not ΔG or mRNA targets determine siRNA activities. Strongest silencing was observed for unstructured sequence L-RC and unstable structures L-US and G-US1 followed by favorable stem-loop structures, however, unstable structure G-US2 failed to induce silencing. Structures G-1-0, L-5-0, and L-0-0 were inactive. IL and 2SL structures showed moderate to good activities although they had no or only few free terminal nts. Their ΔG values allocate to 2 stems which can break up separately. Thus, closed ends of IL and 2SL structures are regarded as pseudo-accessible rather than inaccessible explaining the activity of these miRNA-assigned types of structures. Inhibition of gene expression correlated strongly (r=0.89) with the numbers of free 3′ nts but only moderately (r=0.57) with ΔG (FIG. 2). Thus, regardless of guide strand preferences for sense- or as-siRNAs, structures of as-siRNAs represent major determinants of RNA silencing. In the following we refer to antisense strands when talking about guide-RNA.

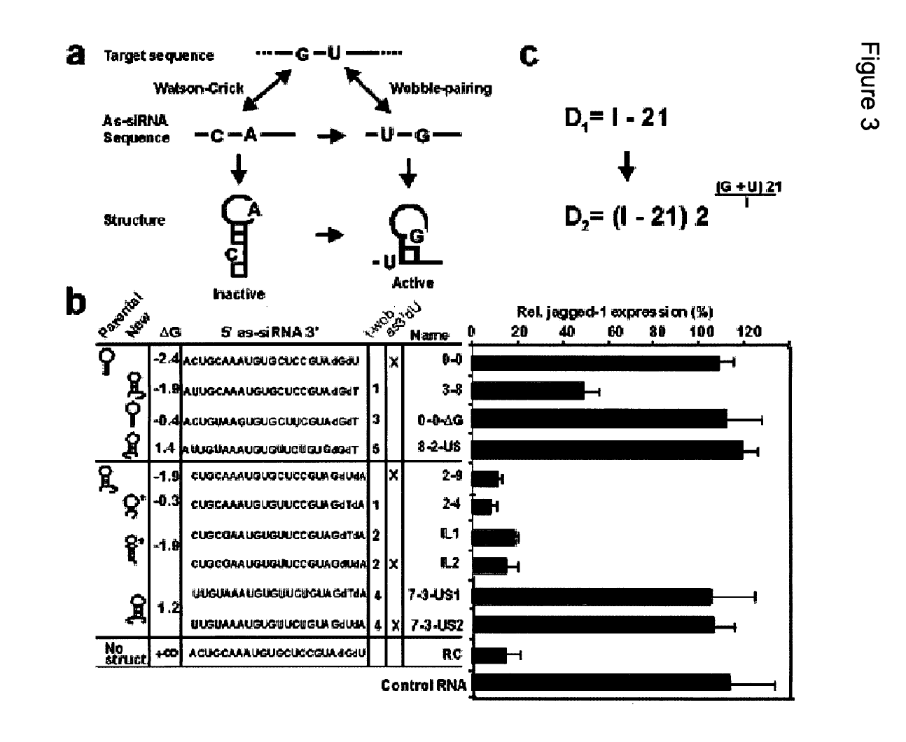

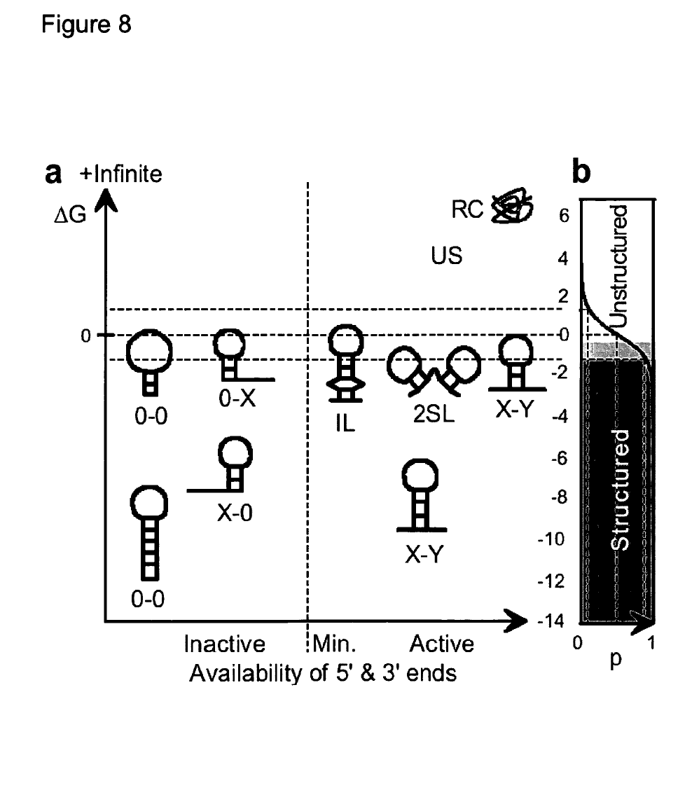

We classify guide-RNA structures as follows: strongest silencing is induced by sequences which do not form secondary structures; second best are stem-loop structures with ≧2 free 5′ and ≧4 free 3′ nts, followed by IL and 2SL structures, and stem-loop structures with short free ends. Stem-loops without free 5′ and/or 3′ nts are inactive indicating that accessible ends provide the condition for activity (see FIG. 8a). Algorithms predict unstable guide-siRNAs with frequencies of ˜25% at physiological salt conditions. If conditions change, such as in the cellular milieu or resulting from interactions with proteins of RISC, unstable foldings may become stable and must not be considered unstructured/active. Independent of the environment, around ΔG=0 (folding probability=½) there is a corridor of uncertainty as to whether structures are folded or not (see Supplementary FIG. 4b online). Only at |ΔG|≧1.3 or ≧2.8 kcal/mol structures are un-/folded with a probability of ≧90 or ≧99%. This may explain that some unstable guide-siRNA structures (G-US1, L-RC, L-US) are active and others (G-US2, 8-2-US, 7-3-US1/2) are not. For the latter, unfavorable mfe structures were predicted. Considering both, activity and predictability of RNA structures, most successful strategies will focus on identification of guide-RNAs which fail to form secondary structures and, secondly, sequences forming favorable mfe structures but no unfavorable suboptimal foldings.

For given mRNA targets of L nts in length, L-21 complementary 21 mer guide strands statistically containing ˜0.14% of most active unstructured sequences are possible. We investigated the possibility to expand the space of complementary guide-siRNAs in order to increase the absolute frequencies of active guide-structures (FIG. 3). We performed A to G (A>G) and C to U (C>U) base exchanges within inactive guide-siRNA 0-0 and active species 2-9 as well as corresponding U>C and G>A exchanges within the sense strands. Such changes preserve target homology, induce wobble base pairing with the target, and can alter guide-RNA structures or ΔG and, hence, silencing activities (FIG. 3a). A C>U exchange at position 2 of the guide-strand changed unfavorable structure 0-0 to favorable structure 3-8 resulting in enhanced silencing (FIG. 3b). A structure-neutral but energy-increasing change from structure 0-0 to structure 0-0-ΔG (3 exchanges) and the change to unstable unfavorable structure 8-2-US (5 exchanges) did not improve the parental molecule indicating that ΔG is not a determinant of RNAi. Changes from structure 2-9 to higher energy structure 2-4 (1 exchange) and internal loop structures IL1 and IL2 (2 exchanges) did not reduce silencing. Structure 2-4 was even more active compared to the parental molecule 2-9. Changes to unstable but unfavorable (only 3 free 3′nts) structures 7-3-US1 and 7-3-US2 resulted in loss of activity. Hence, target-neutral but guide-structure-relevant exchanges can improve active siRNAs or transform inactive species into active ones. The low activity of favorable structure 3-8, the decrease in activity from structure 2-4 to IL1, and possibly the loss of function of unstable structures 7-3-US1 and 7-3-US2 indicate that wobble pairing with the target impairs silencing at 5′ terminal regions of guide strands but is tolerated in a central position of structure 2-4.

This finding is consistent with recent observations with miRNA²². According to equations in FIG. 4c, A>G and C>U exchanges increase the numbers of complementary guide-siRNA by >3 log 10 for target sequences with G/U base contents of 50% allowing accessing new active and more powerful siRNA (see Supplementary Discussion A online). Analogue degenerations are observed among sequences of mature miRNAs²¹ implying that miRNA-activity is modulated by mature miRNA structures.

The observed dependency on free ends of guide-structures has implications for gene silencing. The 5′region was described to determine specificity and binding strength of RISC* whereas central positions and 3′ ends seem to participate in catalysis^{23,24}. The Ago PIWI domain of A. fulgidus and recombinant human Ago-2 anchor guide-RNA 5′-ends which were suggested to initiate nucleation and to determine the distance to mRNA cleavage sites^{25,26}. Free dangling ends of guide-RNA structures appear more flexible and accessible than base-paired ends and more suitable for nucleation or interaction with proteins. The length of free ends of antisense RNA structures was reported to directly correlate with the kinetics of mRNA targeting and with activity²⁷. Guide-strands can be regarded as RISC-associated antisense RNA and we assume that terminal free nts determine the efficiency of mRNA targeting which might be rate-limiting in RNAi. We cannot decide whether mRNA targeting by RISC initiates via 5′ or 3′ ends of guide-siRNA. Empirically, cooperative base pairing after nucleation requires >2 or 3 unpaired nts and our finding that 2 free 5′ nts but >3 free 3′ nts are required for guide-siRNA function favors the idea that mRNA targeting initiates via 3′ ends.

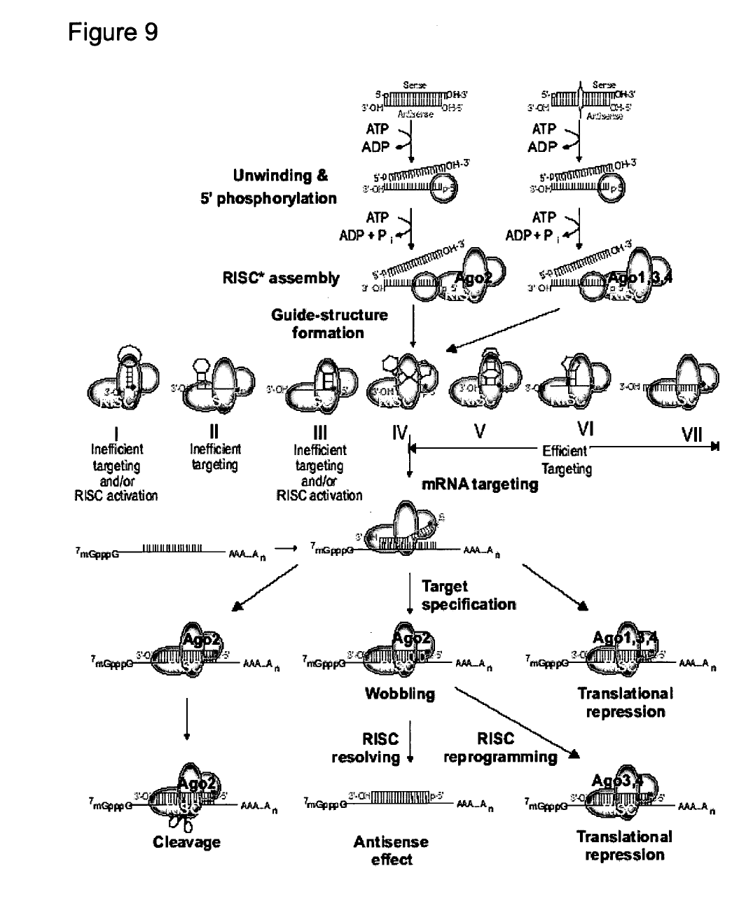

The decision which Argonaute protein is chosen for RISC and which subsequent pathway is initiated must be made before the mRNA is encountered and base matching can be monitored, possibly at the stage of the effector duplex representing a common precursor of the primarily Ago-1-dependent miRNA pathway and Ago-2-dependent siRNA-mediated target cleavage. Silencing activities of duplexes described in FIG. 4 were monitored in Ago-1, Ago-2, and Ago-1+2 knock-down cells (see methods). Silencing induced by mismatched duplexes, i.e. those with a mismatch at antisense strand position 15, was found to be Ago-1-dependent whereas silencing induced by all other duplexes distinguished with Ago-2 knock-down indicating that the structure of the effector duplex determines the choice of the Argonaute protein and, hence, the silencing pathway. The moderate activity of structure 3-8, which gives rise to a single wobble base pair with the target but not within the siRNA duplex, did neither depend on Ago-1 nor on Ago-2. This can be explained if wobble pairing between targets and guide-RNA 5′regions induces reprogramming or resolving of RISC* leading to Ago-1/2-independent silencing or antisense effects (see FIG. 9). Comparisons of duplexes IL2 with IL3 and 2-9 with 2-9-1 and 2-9-2 indicate that duplex-intrinsic base-wobbling and mismatches only marginally impair silencing (FIG. 4).

For computation, guide-RNA is treated like free molecules although they exhibit cellular function only in association with RISC. Such simplification can lead to misinterpretations. In this study, the profound correlations between parameters calculated for isolated guide-RNA and RNAi provide compelling evidence that guide-RNA structures play a crucial role in RNA silencing and can serve as basis for predicting siRNA activity with a resolution at the single-nt-level.

Targets of functional siRNA can coincide with targets of effective antisense oligodeoxyribonucleotides (asODN)¹³⁻¹⁵ and target structure predictions can improve the prediction of site efficacy¹⁶⁻¹⁸. In these studies however, the impact of duplex or as-sRNA-related features on RNAi was not considered. AsODN activity depends on the accessibility of the target structure, which can be predicted by in silico methods³³⁻³⁶. We investigated the impact of target accessibilities on RNAi independent of as-siRNA structures. Using our program TARGETscout we selected a highly accessible target site T-a and an inaccessible site T-i within the jagged-1 mRNA. Target T-a meets the requirements of an accessible site for asODN, i.e. containing a contiguous stretch of ≧10 bases (loop L1) likely of being unpaired. Average accessibility scores SC_{acc} of the targets T (SC_{acc}=21.1%), T-a (SC_{acc}=76.2%), and T-i (SC_{acc}=13.0%), as well as individual scores for each nt within these targets were calculated. Predicted mfe structures and calculated accessibility profiles are shown in FIG. 7). Accessibility scores describe probabilities of local target sites or nts of being unpaired for the complete Boltzmann ensemble of mRNA secondary structures²⁰. High scores reflect targets accessible for hybridization with complementary nucleic acids. To verify target accessibility predictions, antisense oligodeoxyribonucleotides (asODN) corresponding in sequence to selected as-siRNA were tested for gene silencing. Only the sequence of asODN t-a directed against accessible target T-a showed detectable inhibition of target gene expression (see FIG. 7d). The strong differences in siRNA activities which were reflected by the predicted as-siRNA secondary structures were not related to the accessibility scores of the corresponding target sites in T (FIG. 1b and FIG. 7). Favorable as-siRNA structures 6-5, 4-7, and 2-9 each targeting inaccessible local sites in T successfully mediated gene suppression. Conversely, the unfavorable as-siRNA structure t-a (type 0-10) directed against an accessible mRNA target T-a as well as the unfavorable siRNA structures 10-1, 0-0, 0-11, and t-i targeting the inaccessible targets T and T-i, failed to induce RNAi. Thus, as-siRNA structures rather than target structures determine RNAi and accessible target structures are neither necessary nor sufficient for RNAi.

Thermodynamic structure predictions are based on the assumptions that the lowest free energy structure, the minimum free energy (mfe) structure, is the most likely one. Nevertheless, suboptimal foldings can exist and can be relevant as well. Mfe structures and suboptimal folds can be predicted by mfold and other related algorithms. The so called partition function considers all possible folds for a given RNA sequence including the mfe structure and suboptimal foldings as generated by mfold. In many cases partition structures can be drawn from the partition function. If no suboptimal folds occur, the partition structure is equivalent to the mfe structure. For highest congruity between predictions and expected real RNA structures we applied both the partition function and mfold and exclusively selected sequences for which partition structures were equivalent to mfe structures. These structures are depicted figures. That is, for selected RNA sequences no relevant suboptimal foldings exist and it is likely that the mfe structures comply with the real RNA structures. For sequences 2-4, IL1, and IL2, however, suboptimal foldings occurred. In these cases we show partition structures which consider both mfe and suboptimal structures. In our examples, partition structures are highly compatible with observed siRNA activities.

G and U bases can form Watson-Crick and Wobble-base pairs. Consequently, sequences generated by A->G and C->U base exchanges are more competent in forming secondary structures compared to parental sequences and contain a smaller fraction of most active unstructured RNA. Furthermore, not all base exchanges may be tolerated during RNAi. Thus, as-siRNA sequences generated by base exchanges are expected to contain less than 0.14% of highly active species as calculated for random sequences. Nonetheless, the base-exchange technique dramatically increases the numbers of complementary guide strands allowing to access new active and more powerful siRNA.

It has been speculated that stable internal fold-back structures of guide-strands may exist in equilibrium with the duplex form, reducing the effective concentration and activity of siRNA¹¹. Thus, observed structure-function relationships could be crucial for siRNA duplex formation in vitro and/or in vivo. On that level, structures of sense- and as-siRNA would be on a par and one would assume equivalent relations between structures of sense-siRNA and RNAi. Such correlations were not observed. SiRNA activities do not correlate with Δ (r=0.15), free 3′ (r=0.26) or free 5′ nts (r=0.17) of sense-siRNA structures, indicating that structures of as-siRNA play no role in the formation of effector duplexes in vivo. The quality of siRNA duplexes was monitored using a bioanalyzer and did not provide any evidence that guide-RNA fold-back structures impair duplex formation in vitro.

3′ dT overhangs are standard in siRNA synthesis but difficult to consider by RNA folding algorithms which are based on the ribo-alphabet. Uracil but not Thymin can pair with Guanin and the decision of using dT or T instead of dU or U overhangs can alter guide structures if terminal dU/U was predicted to pair with G. In this study, 3′terminal dU/U was only substituted by dT/T if no impact on guide-structures was to be expected. The comparison of structures IL1 with IL2 (FIG. 4) and of structure 7-3-US1 with 7-3-US2 (FIG. 3) did not indicate any difference between 3′ as dT and dU overhangs in our set of structures.

Further data show that programming of RNA silencing pathways may be effected by the structures of siRNA double strands (see FIG. 10). Wobble-base pairs within central regions of the guide strands mediate Ago-2 dependent gene silencing, wherein wobble base pairing between the target and the 5′-terminus of the antisense strands prevents both Ago-1 and Ago-2 dependency. Mismatches within siRNA duplexes result in Ago-1-dependent silencing.

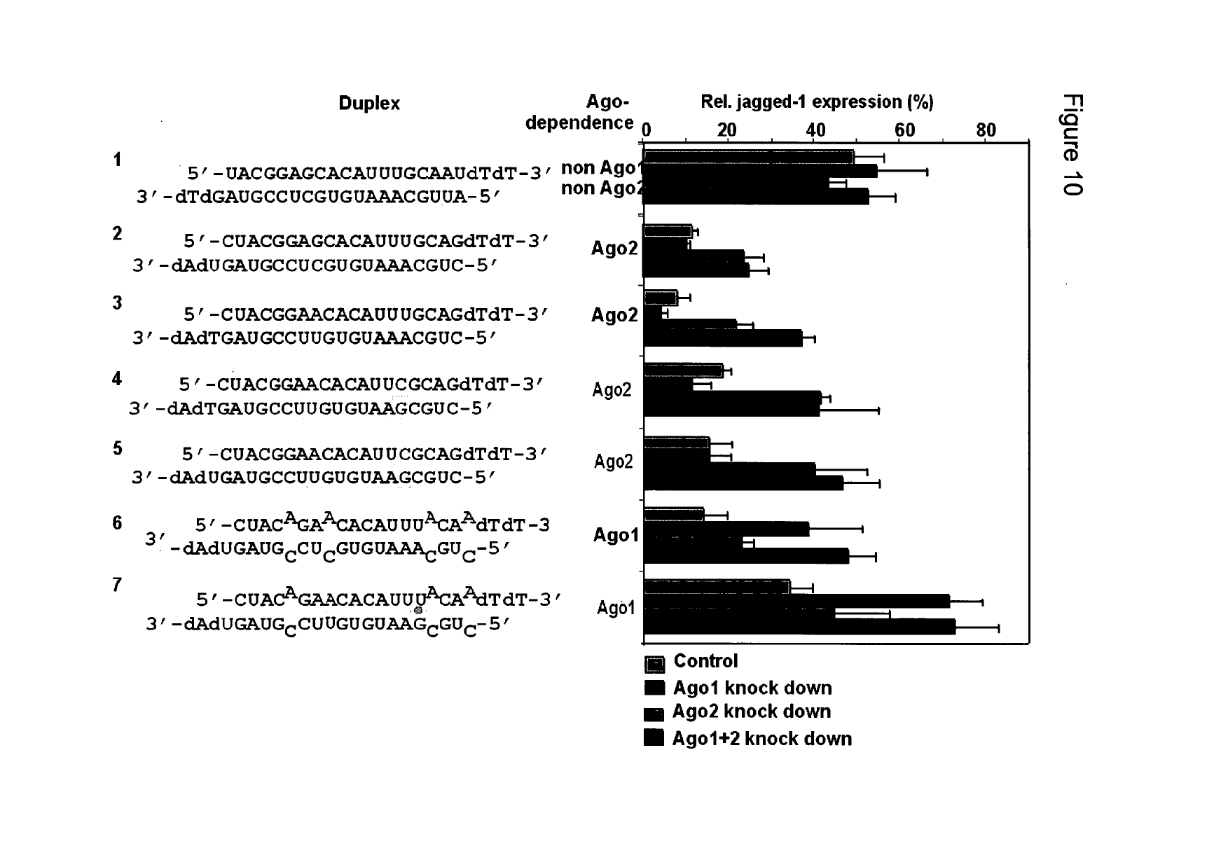

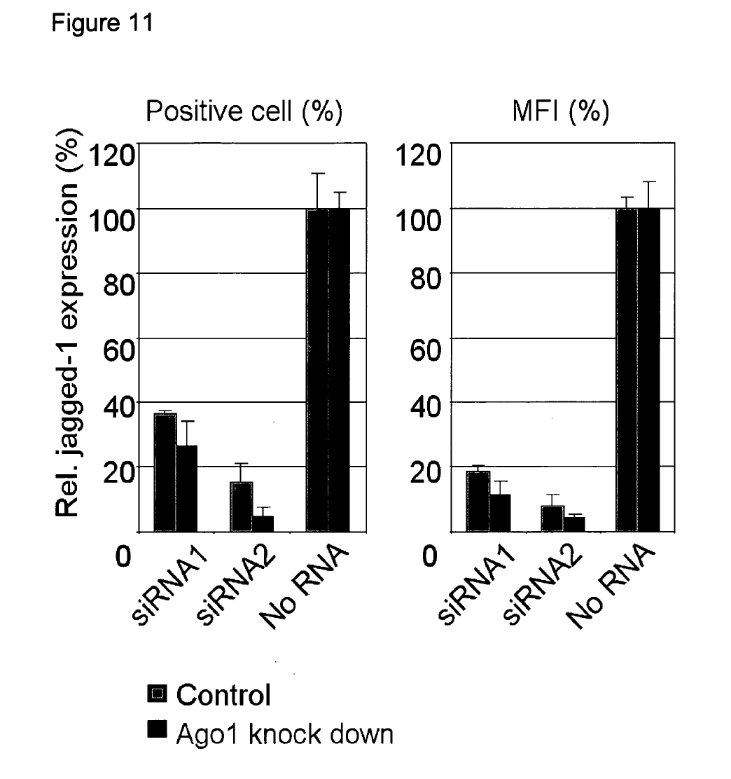

A knockdown of the Ago-1-dependent silencing pathway by co-delivery of Ago-1-specific siRNA and jagged-1 specific siRNA enhances total RNA silencing (see FIG. 11). Ago-1-dependent siRNA directed against Ago-1 is more effective than Ago-2-dependent siRNA directed against Ago-1. Ago-2-dependent siRNA directed against Ago-2 decreases silencing activity (see FIG. 12).

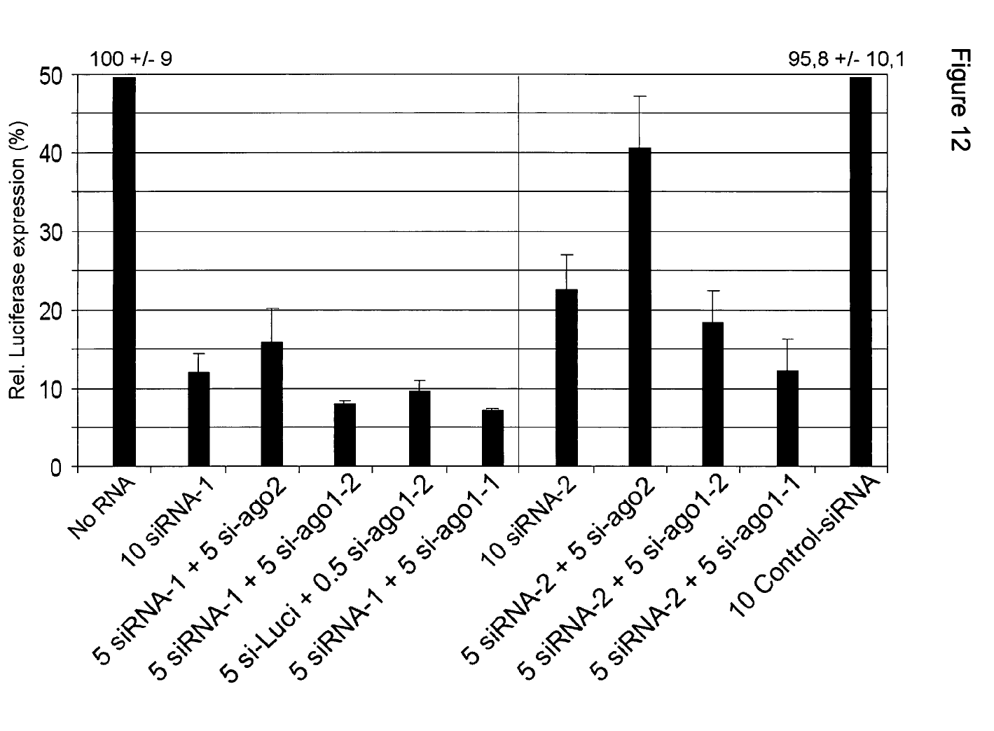

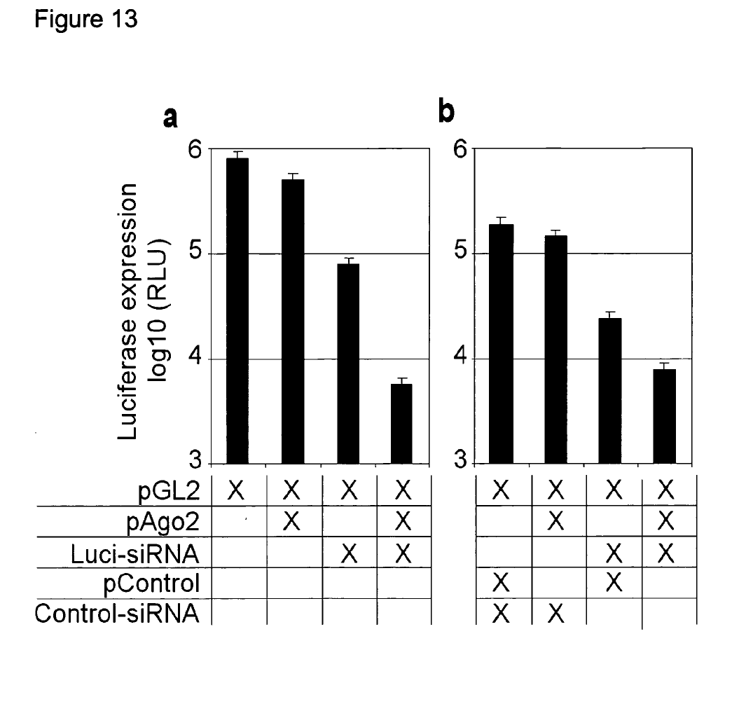

Over-expression of proteins of the silencing machinery, e.g. Ago-1, enhances specific siRNA mediated gene silencing in human tissue culture cells (FIG. 13).

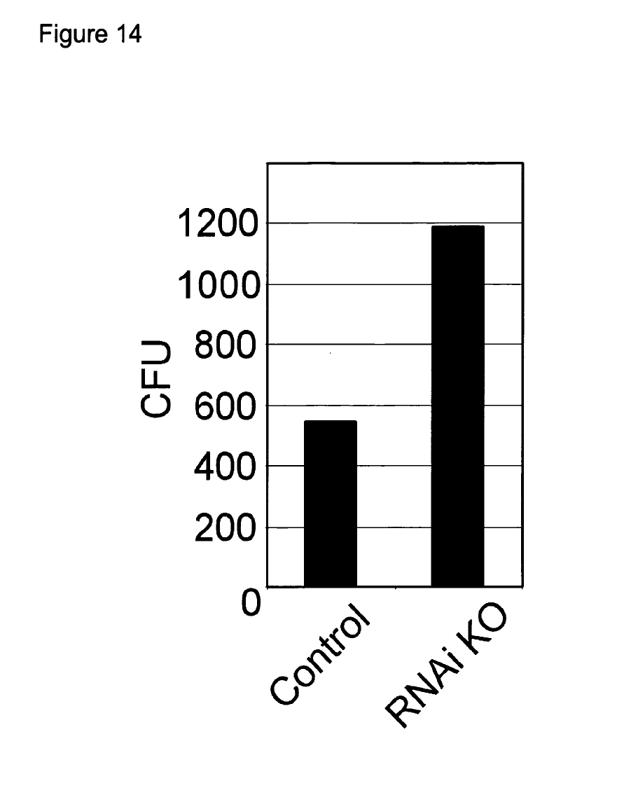

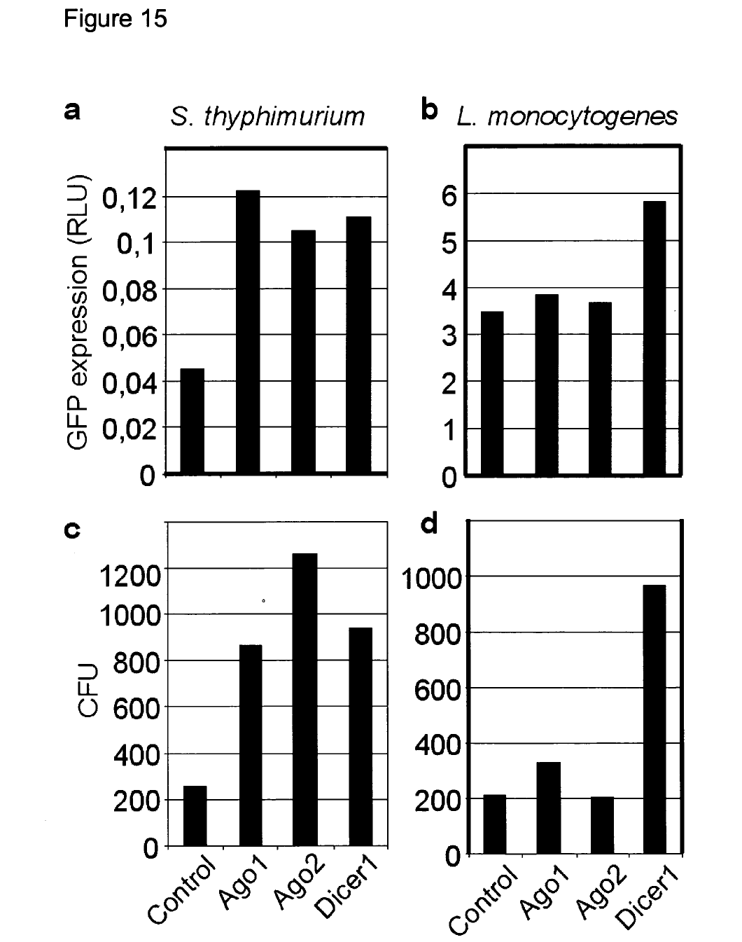

Knocking down expression of proteins involved in the RNA silencing machinery such as for example Ago-1, Ago-2, and/or Dicer1 using gene-specific siRNAs disables RNAi and results in increased susceptibility of human tissue culture cells to microbial (bacterial) pathogens. Conversely, over-expression of proteins of the silencing machinery protects human tissue culture cells from microbial (bacterial) infection (FIG. 14). Thus, RNAi defends human tissue culture cells from microbial (bacterial) invasion or mediates defence.

Susceptibility of human tissue culture cells to S. thyphimurium increases with knock-down of Ago-1, Ago-2, and Dicer1. Susceptibility of human tissue culture cells to L. monocytogenes increases strongly with siRNA-mediated knock-down of Dicer1 and slightly with Ago-1 knock-down (FIG. 13).

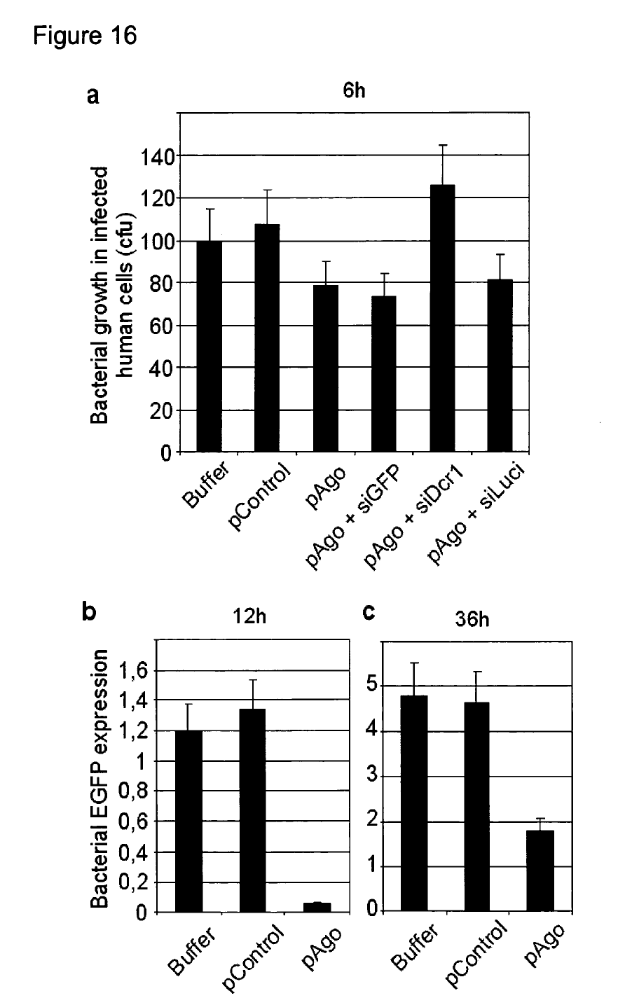

Susceptibility of human tissue culture cells to S. thyphimurium decreases with over-expression of Argonaute-2 protein (pAgo). A control plasmid carrying the same promoter has no effect (pControl). Concomitant siRNA-mediated knock-down of Dicer1 (siDcr1) annuls this effect resulting even in increased susceptibility. Control siRNAs directed against EGFP (siGFP) and Luciferase (siLuci) have no effect. Susceptibility of human tissue culture cells to L. monocytogenes increases strongly with knock-down of Dicer1 and slightly with Ago-1 knock-down (FIG. 16).

### REFERENCES

1. Elbashir, S. M. et al., Duplexes of 21-nucleotide RNAs mediate RNA interference in cultured mammalian cells. Nature 411, 494-498 (2001).
2. Lagos-Quintana, M., Rauhut, R., Lendeckel, W. & Tuschl, T. Identification of novel genes coding for small expressed RNAs. Science 294, 853-858 (2001).
3. Lau, N. C., Lim, L. P., Weinstein, E. G. & Bartel, D. P. An abundant class of tiny RNAs with probable regulatory roles in Caenorhabditis elegans. Science 294, 858-862 (2001).
4. Lee, R. C. & Ambros, V. An extensive class of small RNAs in Caenorhabditis elegans. Science 294, 862-864 (2001).
5. Meister, G. et al. Human Argonaute2 mediates RNA cleavage targeted by miRNAs and siRNAs. Mol. Cell 15, 185-197 (2004).
6. Fagard, M., Boutet, S., Morel, J. B., Bellini, C. & Vaucheret, H. Ago-1, QDE-2, and RDE-1 are related proteins required for post-transcriptional gene silencing in plants, quelling in fungi, and RNA interference in animals. Proc. Natl. Acad. Sci. U.S.A. 97, 11650-11654 (2000).
7. Hammond, S. M., Boettcher, B., Caudy, A. A., Kobayashi, R. & Hannon, G. J. Argonaute2, a link between genetic and biochemical analyses of RNAi. Science 293, 1146-1150 (2001).
8. Tomari, Y., Matranga, C., Haley, B., Martinez, N. & Zamore, P. D. A protein sensor for siRNA asymmetry. Science 306, 1377-1380 (2004).
9. Khvorova, A., Reynolds, A. & Jayasena, S. D. Functional siRNAs and miRNAs exhibit strand bias. Cell 115, 209-216 (2003).
10. Schwarz, D. S. et al. Asymmetry in the assembly of the RNAi enzyme complex. Cell 115, 199-208 (2003).
11. Reynolds, A. et al. Rational siRNA design for RNA interference. Nature Biotechnol. 22, 326-330 (2004).
12. Ui-Tei, K. et al. Guidelines for the selection of highly effective siRNA sequences for mammalian and chick RNA interference. Nucleic Acids Res. 32, 936-948 (2004).
13. Kretschmer-Kazemi Far, R. & Sczakiel, G. The activity of siRNA in mammalian cells is related to structural target accessibility: a comparison with antisense oligonucleotides. Nucleic Acids Res. 31, 4417-4424 (2003).
14. Bohula, E. A. et al. The efficacy of small interfering RNAs targeted to the type 1 insulin-like growth factor receptor (IGF1R) is influenced by secondary structure in the IGF1R transcript. J. Biol. Chem. 278, 15991-15997 (2003).
15. Vickers, T. A. Efficient reduction of target RNAs by small interfering RNA and RNase H-dependent antisense agents. A comparative analysis. J. Biol. Chem. 278, 7108-7118 (2003).
16. Heale, B. S. E., Soifer, H. S., Bowers, C. & Rossi, J. J. SiRNA target site secondary structure predictions using local stable substructures. Nucleic Acids Res. 33, 30e (2005).
17. Schubert, S., Grunweller, A., Erdmann. V. A. & Kurreck, J. Local RNA target structure influences siRNA efficacy: systematic analysis of intentionally designed binding regions. J. Mol. Biol. 348, 883-893 (2005).
18. Overhoff, M., Alken, M., Far, R. K., Lemaitre, M., Lebleu, B., Sczakiel, G. & Robbins, I. Local RNA target structure influences siRNA efficacy: a systematic global analysis. J. Mol. Biol. 348, 871-881 (2005).
19. Zuker, M. & Stiegler, P. Optimal computer folding of large RNA sequences using thermodynamics and auxiliary information. Nucleic Acids Res. 9, 133-148 (1981).
20. McCaskill, J. S. The equilibrium partition function and base pair binding probabilities for RNA secondary structure. Biopolymers 29, 1105-1119 (1990).
21. Griffiths-Jones, S. The microRNA Registry Nucleic Acids Res. 32, Database issue, D109-111 (2004).
22. Doench, J. G. & Sharp, P. A. Specificity of microRNA target selection in translational repression. Genes Dev. 18, 504-511 (2004).
23. Haley, B. & Zamore, P. Kinetic analysis of the RNAi enzyme complex. Nature Struct. Mol. Biol. 11, 599-606 (2004).
24. Schwarz, D. S., Hutvágner, G., Haley, B. & Zamore, P. D. Evidence that siRNAs function as guides, not primers, in the Drosophila and human RNAi pathways. Mol. Cell 10, 537-548 (2002).
25. Ma, J.-B. et al. Structural basis for 5′-end-specific recognition of guide RNA by the A. fulgidus Piwi protein. Nature 434, 666-670 (2005).
26. Rivas, F. V. et al. Purified Argonaute2 and an siRNA form recombinant human RISC. Nat. Struct. Biol. 12, 340-349 (2005).
27. Patzel, V. & Sczakiel, G. Theoretical design of antisense RNA structures substantially improves annealing kinetics and efficacy in human cells. Nature Biotechnol. 16, 64-68 (1998).
28. Mathews, D. H., Burkard, M. E., Freier, S. M., Wyatt, J. R. & Turner, D. H. Predicting oligonucleotide affinity to nucleic acid targets. RNA 5, 1458 (1999).
29. Hofacker, I. L. Vienna RNA secondary structure server. Nucleic Acids Res. 31, 3429-3431 (2003).
30. Lee, N. S. Et al. Expression of small interfering RNAs targeted against HIV-1 rev transcripts in human cells. Nature Biotechnol. 20, 500-505 (2002).
31. Heinonen, J. E., Smith, C. I. & Nore, B. F. Silencing of Bruton's tyrosine kinase (Btk) using short interfering RNA duplexes (siRNA). FEBS Lett. 527, 274-278 (2002).
32. Holen, T., Amarzguioui, M., Wiiger, M. T., Babaie, E. & Prydz, H. Positional effects of short interfering RNAs targeting the human coagulation trigger Tissue factor. Nucleic Acids Res. 30, 1757-1766 (2002).
33. Patzel, V., Steidl, U., Kronenwett, R., Haas, R. & Sczakiel, G. A theoretical approach to select effective antisense oligodeoxyribonucleotides at high statistical probability. Nucleic Acids Res. 27, 4328-4334 (1999).
34. Scherr, M., Rossi, J. J., Sczakiel, G. & Patzel, V. RNA accessibility prediction: a theoretical approach is consistent with experimental studies in cell extracts. Nucleic Acids Res. 28, 2455-2461 (2000).
35. Ding, Y. & Lawrence, C. E. Statistical prediction of single-stranded regions in RNA secondary structure and application to predicting effective antisense target sites and beyond. Nucleic Acids Res. 29, 1034-1046 (2001).
36. Giddings, M. C. et al. Artificial neural network prediction of antisense oligodeoxynucleotide activity. Nucleic Acids Res. 30, 4295-4304 (2002).

1. A method for preparing a double stranded RNA molecule with target gene specific silencing activity, comprising the steps
(a) identifying a double stranded RNA molecule directed to the mRNA of a target gene, wherein said RNA molecule comprises:
(i) a double stranded portion of 9-35 nucleotides and optionally at least one 3′-overhang,
(ii) an antisense strand which has a sufficient degree of complementarity to the mRNA of the target gene for formation of an RNA-induced silencing complex (RISC), and accessible 5′- and 3′-ends which do not form stable intramolecular secondary structures,
(iii) a sense strand which has a sufficient degree of complementarity to the antisense strand for interaction with a RISC, and
(b) preparing a double stranded RNA molecule or a precursor thereof or a DNA molecule encoding said RNA molecule or precursor.

2. The method according to claim 1 wherein the target gene specific silencing activity is a transcriptional gene silencing (TGS) activity or a post-transcriptional gene silencing (PTGS) activity preferably selected from RNA interference and/or translational attenuation.

3. The method according to any of claims 1 or 2 wherein the structure of the 5′- and 3′-ends or the antisense strand have an unpaired conformation, an internal loop (il) conformation or a two-stem-loop (2sl) conformation.

4. The method according to any of claims 1-3 wherein the structure of the antisense strand has a minimal Gibbs free energy of about ≧0 kcal/mol, preferably of about ≧0.5 kcal/mol, more preferably of about ≧1.3 kcal/mol and most preferably of about ≧2.8 kcal/mol.

5. The method according to any of claims 1-4 wherein the antisense strand is substantially free from secondary structure and comprises a random coil structure.

6. The method according to any of claims 1-5 wherein the 5′- and 3′-endfs of the antisense strand are accessible as determined by a partition function approach which gives base-pairing probabilities for a Boltzmann ensemble of secondary structures.

7. The method according to any of the claims 1-6 wherein said double stranded RNA molecule is a siRNA or a miRNA.

8. The method according to any one of claims 1-7 wherein the length of the antisense strand and the sense strand, respectively, is between 15 and 30 nucleotides, preferably between 19 and 25 nucleotides.

9. The method according to any one of claims 1-8 wherein the double stranded RNA molecule comprises a double stranded portion and at least one 3′-overhang of from 1-10, preferably from 1-5 nucleotides.

10. The method according to any one of claims 1-9, wherein said antisense and/or said sense strand of the double stranded RNA molecule comprise at least one modified nucleotide analogue.

11. The method according to any one of claims 1-10, wherein said modified nucleotide analogue is selected from sugar-, backbone- and nucleobase modified nucleotides and combinations thereof.

12. The method according to any one of claims 1 to 11 wherein the double stranded RNA molecule further comprises 5′- and/or 3′-modifications preferably selected from lipid groups, e.g. cholesterol groups and vitamins.

13. The method according to any one of claims 1 to 12, wherein said sense and said antisense strand are chemically and/or enzymatically synthesized.

14. The method of any of claims 1 to 13, wherein the antisense strand is completely complementary to said target RNA, wherein complementarity comprises Watson-Crick and base pairs and Wobble (G-U, U-G) base pairs.

15. A method according to any of claims 1 to 14, wherein the length of the 5′-accessible end is at least 2, 3, or 4 nucleotides.

16. The method of any of the claims 1 to 15, wherein the length of the 3′-accessible and is at least 4, 5, 6, 7, 8, 9 or 10 nucleotides.

17. The method according to any one of claims 1 to 16, wherein step (b) comprises synthesizing the antisense RNA strand and the sense RNA strand and combining the strands to form the double stranded RNA molecule.

18. The method according to any one of claims 1-16, wherein step (b) comprises synthesizing the precursor of the double stranded RNA molecule and subjecting the precursor to a processing step whereby the double stranded RNA molecule is formed.

19. The method according to any one of claims 1 to 16, wherein step (b) comprises synthesizing the DNA molecule encoding the double stranded RNA molecule or the precursor thereof and transcribing the DNA molecule whereby the double stranded RNA molecule or the precursor thereof is formed, and wherein the precursor is subjected to a processing step whereby the double stranded RNA molecule is formed.

20. The use of a double stranded RNA molecule obtainable by a method according to any of the claims 1 to 19 for the manufacture of a reagent, a diagnostic or a medicament.

21. A method for regulating the expression of a target gene in a cell, an organism or in a cell-free system, comprising the steps of
(a) preparing a double stranded RNA molecule according to any of claims 1 to 19 or a precursor thereof, or a DNA molecule encoding the double stranded RNA molecule or the precursor thereof, and
(b) contacting the module of (a) with said cell, organism or cell-free system under conditions under which target-specific gene silencing occurs.

22. A method regulating the expression of a target gene in a cell, an organism or a cell-free system comprising the steps of:
(a) identifying a double a double stranded RNA molecule directed to the mRNA of a target gene, wherein said RNA molecule comprises:
(i) a double stranded portion of 9-35 nucleotides and optionally at least one 3′ overhang,
(ii) an antisense strand which has a sufficient degree of complementarity to the mRNA of the target gene for formation of an RNA-induced silencing complex (RISC), and accessible 5′- and 3′-ends which do not form stable intramolecular secondary structures,
(iii) a sense strand which has a sufficient degree of complementarity to the antisense strand for interaction with a RISC,
(b) preparing a double stranded RNA molecule or a precursor thereof or a DNA molecule encoding said RNA molecule or precursor and
(c) contacting said mRNA of the target gene with said double stranded RNA molecule under conditions under which target-specific nucleic acid silencing occurs.

23. A method of producing a cell, non-human organism or cell-free system, comprising the steps:
(a) preparing a double stranded RNA molecule according to any of claims 1 to 19 or a precursor thereof, or a DNA molecule encoding the double stranded RNA molecule or the precursor thereof, and
(b) introducing the molecule of (a) into said cell, organism or cell-free system under conditions under which target-specific gene silencing occurs.

24. A knockdown cell, non-human organism or cell-free system obtainable by the method of claim 23.

25. A method of examining the function of a target gene in a cell or non-human organism or a cell-free system comprising:
(a) preparing a double stranded RNA molecule according to any of claims 1 to 19 or a precursor thereof, or a DNA molecule encoding the double stranded RNA molecule or the precursor thereof, and
(b) introducing the molecule of (a) into said cell, organism or cell-free system under conditions under which target-specific gene silencing occurs, and/or system of (b), and
(c) observing the phenotype of the cell, organism or system of (b) and optionally, comparing said phenotype to that of an appropriated control cell, organism or system.

26. A reagent, a diagnostic or a medicament comprising a double stranded RNA molecule obtainable by a method of any one of the claims 1 to 19.

27. A method for preparing a double stranded RNA molecule with a target gene specific gene silencing activity, comprising the steps of:
(a) identifying a double stranded RNA molecule directed to the mRNA of a gene, wherein said RNA molecule comprises:
(i) a double stranded portion 9-35 nucleotides and optionally at least one 3′ overhang,
(ii) an antisense strand which has a sufficient degree of complementarity to the mRNA target gene for formation of an RNA-induced silencing complex (RISC), and accessible 5′- and 3′-ends which do not form stable intramolecular secondary structures, and at least one wobble base pair (G-U, U-G) between the antisense strand and the target sequence,
(iii) a sense strand which has a sufficient degree of complementarity to the antisense strand for interaction with a RISC, and
(b) preparing a double stranded RNA molecule or a precursor thereof or a DNA molecule encoding said RNA molecule or precursor.

28. The method of claim 27 wherein the wobble base pair is located in the antisense strand at a position selected from positions 3, 4, 5, 6, 7, 8, 9, 10, 11, 12, 13, 14, 15, 16, 17, 18, 19 and 20, located within the double stranded portion of the RNA molecule.

29. A double stranded RNA molecule with target gene specific silencing activity comprising:
(a) a double stranded portion of 9-35 nucleotides and optionally at least one 3′-overhang
(b) an antisense RNA strand which has a sufficient degree of complementarity to the mRNA of the target gene for formation of an RNA-induced silencing complex (RISC), and accessible 5′- and 3′-ends which do not form stable intramolecular secondary structures, and
(c) a sense RNA strand which has a sufficient degree of complementarity to the antisense strand for interaction with a RISC, or a precursor thereof or a DNA molecule encoding the double stranded RNA molecule or the precursor thereof.

30. A double stranded RNA molecule with target gene specific silencing activity comprising a double stranded portion:
(a) an antisense RNA strand which has 9-35 nucleotides and optionally at least one 3′-overhang,
(b) a sufficient degree of complementarity to the mRNA of the target gene for formation of an RNA-induced silencing complex (RISC), accessible 5′- and 3′-ends which do not form stable intramolecular secondary structures, and at least one wobble base pair (G-U, U-G) between the antisense strand and the target sequence, and
(c) a sense RNA strand which has a sufficient degree of complementarity to the antisense strand for interaction with a RISC, or a precursor thereof or a DNA molecule encoding the double stranded RNA molecule or the precursor thereof.

31. The molecule of claim 29 or 30 which is directed against a target gene selected from pathogen genes, mammalian endogenous genes, and reporter genes.

32. The molecule of any of claims 29 to 31 wherein said RNA molecule has a target gene silencing activity of at least 90%, 92%, 96% or 98%.

33. The molecule of any of claims 29-32 which is a reagent, a diagnostic or a medicament.

34-97. (canceled)
